(19) United States
(12) Patent Application Publication
(10) Pub. No.: US 2026/0201442 A1
(43) Pub. Date: Jul. 16, 2026

(54) MULTI-MODAL, SAME-CELL ASSAY FOR SEQUENCING AND PROTEIN DETECTION

(71) Applicants: CZ BIOHUB SF, LLC, San Francisco, CA (US); THE REGENTS OF THE UNIVERSITY OF CALIFORNIA, Oakland, CA (US)

(72) Inventors: Amy E. Herr, Berkeley, CA (US); Trinh Lam, San Francisco, CA (US); Anna Fomitcheva Khartchenko, Oakland, CA (US)

(21) Appl. No.: 19/423,111

(22) Filed: Dec. 17, 2025

Related U.S. Application Data
(60) Provisional application No. 63/736,438, filed on Dec. 19, 2024.

(51) Int. Cl. C12Q 1/6806; B01L 3/00

(52) U.S. Cl. CPC C12Q 1/6806; B01L 3/502761; B01L 2200/025; B01L 2200/0647; B01L 2200/14; B01L 2300/12; B01L 2400/0415; B01L 2400/049

(57) ABSTRACT

Disclosed herein are apparatuses and methods relating to single-cell organelle extraction with cellular indexing. In an implementation, an example multilayer microfluidic device includes a plurality of first microwells and a plurality of second microwells. The first microwells include a gel layer and a gel floor layer. Each of the first microwells is aligned with one of the second microwells in the implementation shown. One or more single cells may be lysed within the first microwells in operation to release cytoplasmic biomolecules and organelles and an electric field may be applied to the first microwell in the gel layer, thereby separating cytoplasmic biomolecules within the gel layer and the gel floor layer is dissolved to enable organelles to be collected in the individual second microwells. The gel floor layer may be dissolved by contacting the gel floor with a reducing agent as an example.

### CROSS-REFERENCE TO RELATED APPLICATIONS

This application claims priority to U.S. Provisional Application No. 63/736,438 filed Dec. 19, 2024. The entire contents of which is fully incorporated herein by reference.

### STATEMENT REGARDING FEDERALLY SPONSORED RESEARCH

This invention was made with government support under Grant Number CA203018 awarded by the United States Institutes of Health. The government has certain rights in the invention.

### BACKGROUND OF THE INVENTION

Cells are composed of specialized organelles that each perform unique functions. Structural and functional assays rely on organelle isolation, wherein the integrity of the isolated organelles directly affects analysis accuracy, ultimately shaping our understanding of biology [1]. Bulk organelle-fractionation methods (e.g., density-gradient centrifugation, immune-isolation, free-flow electrophoresis, detergent-based chemical fractionation, enzymatic digestion) are labor-intensive, designed for pooled cell suspensions and not suitable for sparingly available specimens, and offer low organelle-recovery yields. Although suffering from these performance shortcomings, density-gradient centrifugation remains widely used [2-4]. Immuno-isolation is constrained by the availability and quality of antibody probes specific to organelle surface proteins [5]. Free-flow electrophoresis separates cellular organelles [6, 7] with low recovery purity and resolution [8]. Detergent cocktails enrich specific cellular fractions; with each chemical component having a distinct solubilization efficiency [9, 10]. Yet, enzymatic treatments are known to perturb cell-cycle status, apoptosis, and structural alterations [11, 12]. Overall, bulk organelle-isolation methods require multiple steps requiring extensive manual handling and yield compromised purity and integrity of the isolated organelles, thus impacting functional analysis.

Microfluidic technologies offer enhanced precision in organelle isolation from sparingly available starting samples and can overcome limitations of isolated-organelle yield and sample-prep throughput. These tools include techniques utilizing magnetic nanoparticles [13], immuno-affinity [14, 15], flow-based or channel structures [16-19], digital microfluidics [20, 21], magnetophoretic-based microfluidics [22], and devices structured to capture DNA [23, 24]. While precise, multi-step process flows (e.g., on-chip extraction, isolation, and off-chip recovery) can be a source of organelle damage and yield loss.

Even with the advent of precision tools for organelle isolation, the post-isolation pooling of isolated organelles remains ubiquitous, making even contemporary microfluidic techniques incompatible with follow-on single-cell or single-organelle analyses that require indexing of organelle back to the originating cell. Indexing an isolated organelle to the originating cell forms a basis for understanding organelle-derived heterogeneity that exists between cell types and among individual cells, even of the same type [25]. In a related aspect of performance: the preservation of spatial information is increasingly sought, such as mapping an isolated organelle(s) back to the originating tissue context. Logically, mapping back to the single originating cell is also sought because functional links between biological processes can (and do) occur at the level of single cells.

An active area of organelle-and cellular-level biology is the study of the nucleus as a coordinating—and typically the largest—cellular organelle. Microfluidic tools make single-nucleus measurements possible. To analyze chromosomal DNA from single nuclei, Benítez et al. introduced a micropillar array and hydrodynamic flows to extract and stretch chromosomal DNA from ~100 single mammalian cells per chip [26]. Following these precise, in-situ assays of chromosomal-DNA stretching from a single cell, DNA was recovered and quantified off chip. Similarly, Wang et al. utilized microchannel geometries to isolate and stretch chromosomal DNA from 10-20 nuclei for subsequent DNA fluorescence in-situ hybridization (FISH), with each signal traced back to its originating nucleus [27]. While limited to assessing DNA damage, conventional agarose-slab embedded and microwell-based comet assays, do allow researchers to assess DNA damage and map back to originating cell [28]. These existing techniques point to the promise for assessing other nuclear components—including proteins such as transcription factors—and mapping said measurements back to the originating cell and/or tissue context. Current multimodal assays, including those relying on commercial systems, are limited in their ability to detect surface or intra-nuclear proteins and require prior knowledge of target proteins to design specific antibody-oligo conjugates. Additionally, these assays typically require nuclei fixation, which may limit subsequent analyses, and they have not detected or discussed protein isoforms. This presents a challenge when an unexpected finding arises from next-generation sequencing (NGS), as there is no way to retrospectively assess corresponding protein expression.

Disclosed herein are apparatuses, assays, and methods of use that are configured to isolates single cells in polyacrylamide gel microwells, separate cellular compartments for sequencing, and spatially preserve proteins for future analysis such as single cell Western blot or single cell mass spectrometry.

### SUMMARY OF THE INVENTION

Disclosed herein is a method for isolating cytoplasmic biomolecules and organelles from a single cell in separate chambers, the method comprising: isolating one or more single cells in individual first microwells, wherein said first microwells comprise a gel layer and a gel floor layer; lysing the one or more single cells to release cytoplasmic biomolecules and organelles; applying an electric field to the first microwell in the gel layer, thereby separating cytoplasmic biomolecules within the gel layer; collecting organelles in individual second microwells; said collecting comprising (i) dissolving the gel floor layer and (ii) applying a vacuum force; thereby isolating cytoplasmic biomolecules and organelles from a single cell in separate chambers.

In some embodiments, isolating of the one or more single cells step in (a) comprises depositing one or more single cells into individual microwells from a microfluidic channel. In some embodiments, said gel layer comprises one or more of Bis/acrylamide monomer, a photoinitiator, a UV photoimmobilizer, a buffer, and water. In some embodiments, the photoinitiator is VA-086, said UV photoimmobilizer is BPMA, and said buffer comprises Tris-HCl. In some embodiments, Bis-acrylamide monomer is Acrylamide/Bis-acrylamide 40% solution (29:1). In some embodiments, said Bis gel is approximately 60 μm in height and comprises microwells approximately 40 μm in diameter. In some embodiments, said gel floor layer comprises one or more of a reversible crosslinker, acrylamide, TEMED, and APS. In some embodiments, said reversible crosslinker is N,N′-Bis(acryloyl)cystamine (BAC) gel. In some embodiments, said BAC is present at a concentration of about 0.150% to 0.400%. In some embodiments, said BAC is present at about 0.200%. In some embodiments, said gel floor layer is approximately 50 μm in height. In some embodiments, said gel floor can be dissolved by contacting said gel floor with a reducing agent.

In some embodiments, the lysing of step (b) comprises contacting the one or more single cells with a chemical lysing agent. In some embodiments, the chemical lysing agent comprises a buffer comprising a differential detergent fractionation (DDF) agent. In some embodiments, the DDF agent is selected from the group consisting of Digitonin, and Triton-X. In some embodiments, the buffer comprising the DDF agent lyses cytoplasmic membranes but leaves organellar membranes intact.

In some embodiments, the electric field is applied under conditions that allow separation of the cytoplasmic biomolecules based on molecular weight (MW) and/or chemical properties. In some embodiments, the second microwell comprises an elastomer. In some embodiments, said elastomer is selected from the group consisting of PDMS and a curing agent (10:1). In some embodiments, the second microwell comprises PDMS and is approximately 200 μm in height and comprises microwells approximately 250 μm by 350 μm.

In some embodiments, said dissolving the gel floor comprises contacting the gel floor with a reducing agent. In some embodiments, said reducing agent is selected from the group consisting of dithiothreitol (DTT). In some embodiments, dissolving of the gel floor is complete in approximately 3-5 minutes.

In some embodiments, said applying the vacuum force comprises a vacuum force selected from the group consisting of about 30 mbar to about 130 mbar. In some embodiments, said vacuum force does not cause the second microwell to collapse.

In some embodiments, said second microwell comprises barcoded beads. In some embodiments, said barcoded beads and said second microwells comprise oligonucleotide primers capable of hybridizing to nucleic acids of the organelles. In some embodiments, said second microwell is loaded with barcodes. In some embodiments, said method further comprises pooling the barcoded beads comprising hybridized nucleic acids for amplification and/or sequencing.

In some embodiments, one or more single cells are selected from the group consisting of mammalian cells, human cells, tumor cells, organoids, patient-derived cells or patient-derived organoids, and dissociated human tissues. In some embodiments, said cytoplasmic biomolecules comprise proteins. In some embodiments, said organelles comprise nuclei and mitochondria.

In some embodiments, said isolating comprises isolating 6, 12, 24, 48, 96, 256, 384 or 1536 single cells in step (a). In some embodiments, said isolating comprises using a stacked microwell pair according to any of the embodiments described herein. In some embodiments, the isolating occurs concurrently. In some embodiments, the isolating comprises using an apparatus comprising a plurality of first microwells, wherein the first microwells comprise a gel layer and a gel floor layer; a plurality of second microwells, each first microwell aligned with one of the second microwells, wherein one or more single cells are to be lysed within the first microwells to release cytoplasmic biomolecules and organelles, wherein an electric field is to be applied to the first microwell in the gel layer, thereby separating cytoplasmic biomolecules within the gel layer, and wherein the gel floor layer is to be dissolved to enable organelles to be collected in the individual second microwells.

Also described herein a method for detecting one or more cytoplasmic biomolecules and one or more organelles from a single cell, said method comprising: isolating cytoplasmic biomolecules and organelles from a single cell; detecting one or more cytoplasmic biomolecules, said detecting comprising any one or more detection methods comprising electrophoresis, immunoprobing and mass spectrometry; detecting one or more organelles, said detecting comprising any one or more detection methods comprising sequencing, imaging, morphology identification, in situ hybridization, and rolling circle amplification; thereby detecting one or more cytoplasmic biomolecules and one or more organelles from a single cell.

In some embodiments, the cytoplasmic biomolecules are covalently fixed within the gel layer prior to detecting. In some embodiments, the cytoplasmic biomolecules are transferred to a membrane prior to detecting.

In some embodiments, the organelles are contacted in the second microwell by oligonucleotides corresponding to primer pair sequences under conditions that allow hybridization of the primer pair to nucleic acids of the organelles, thereby forming a complex, and wherein the complex is subjected to amplification and/or sequencing. In some embodiments, the sequencing is selected from the group consisting of snATAC-seq, scATAC-seq, scRNA-seq, snRNA-seq, DNA methylation, DNA-sequencing, and whole genome sequencing.

In some embodiments, the detecting of the one or more cytoplasmic biomolecules is linked to the one or more organelles from the same single cell.

In some embodiments, the method optionally further comprises the step of capturing one or more images of the cytoplasmic biomolecules and/or organelles.

In some embodiments, the detecting comprises using an apparatus comprising a plurality of first microwells, wherein the first microwells comprise a gel layer and a gel floor layer; a plurality of second microwells, each first microwell aligned with one of the second microwells, wherein one or more single cells are to be lysed within the first microwells to release cytoplasmic biomolecules and organelles, wherein an electric field is to be applied to the first microwell in the gel layer, thereby separating cytoplasmic biomolecules within the gel layer, and wherein the gel floor layer is to be dissolved to enable organelles to be collected in the individual second microwells.

Also disclosed here is a method of barcoding microwells suitable for single cell analyses in a microwell system, the method comprising: (a) labeling magnetic beads with known oligonucleotide barcodes, wherein the oligonucleotide barcodes comprise a bead barcode sequence and a first primer pair sequence; (b) delivering the labeled magnetic beads into microwells using a microfluidic channel of a first microfluidic device; (c) removing the first microfluidic device; (d) applying a magnetic force under conditions that prevent the labeled magnetic beads from escaping the microwells; (e) delivering free oligonucleotides to the microwells comprising the labeled magnetic beads using a microfluidic channel of a second microfluidic device, wherein the free oligonucleotides comprise a free barcode sequence and a second primer pair sequence.

In some embodiments, the magnetic beads comprise an iron oxide core. In some embodiments, the magnetic beads are labeled with the known oligonucleotide barcodes via streptavidin, and biotin. In some embodiments, approximately 5 mg/mL beads are delivered to an individual microwell.

In some embodiments the method optionally further comprises delivering single cells, microorganisms, organoids, spheroids, nucleic acids or organelles to individual microwells comprising the labeled barcoded beads and the free oligonucleotides.

In some embodiments, organelles from single cells are delivered to the microwells. In some embodiments, the labeled magnetic beads are delivered into the microwells at a flow rate of approximately 50-1000 μL/minute.

In some embodiments, the first microfluidic device and the second microfluidic device are microfluidic chips comprising orthogonal channels. In some embodiments, the barcoded microwells comprise an elastomer. In some embodiments, said elastomer is PDMS.

In some embodiments, said second microwells comprise PDMS and are approximately 200 μm in height and comprise microwells approximately 250 μm by 350 μm.

Also disclosed herein is a method of highly parallel, dead-end, vacuum-based reagent delivery to microwells suitable for single cell analyses in a microwell system, the method comprising: bonding a bifurcated delivery membrane to a barcoded PDMS microwell (after organelle transfer to the PDMS microwell); bonding a vacuum suction layer on top of the delivery membrane; and activating a vacuum force to generate negative pressure across the delivery membrane, therefore, driving the reagents loading to each microwell simultaneously.

Also disclosed herein is an apparatus, comprising: a plurality of first microwells, wherein the first microwells comprise a gel layer and a gel floor layer; a plurality of second microwells, each first microwell aligned with one of the second microwells, wherein one or more single cells are to be lysed within the first microwells to release cytoplasmic biomolecules and organelles, wherein an electric field is to be applied to the first microwell in the gel layer, thereby separating cytoplasmic biomolecules within the gel layer, and wherein the gel floor layer is to be dissolved to enable organelles to be collected in the individual second microwells.

In some embodiments, each of the first microwells that is aligned with one of the second microwells form a stacked microwell pair.

In some embodiments, the apparatus further comprises a plurality of stacked microwell pairs, each microwell pair comprising one of the first microwells and one of the second microwells.

In some embodiments, the gel floor is to be dissolved by contacting the gel floor with a reducing agent.

In some embodiments, the gel layer is approximately 60 μm in height and comprises microwells approximately 40 μm in diameter.

In some embodiments, the gel layer comprises one or more of Bis/acrylamide monomer, a photoinitiator, a UV photoimmobilizer, a buffer, and water. In some embodiments, said photoinitiator is VA-086, said UV photoimmobilizer is BPMA, and said buffer comprises Tris-HCl.

In some embodiments, the gel layer comprises a Bis gel comprising acrylamide and bis-acrylamide.

In some embodiments, the Bis gel comprises Acrylamide/Bis-acrylamide 40% solution (29:1).

In some embodiments, the Bis gel comprises Acrylamide/Bis-acrylamide of about 4% to about 12%. In some embodiments, the Bis gel comprises Acrylamide/Bis-acrylamide of about 7%. In some embodiments, the gel floor layer comprises a reversible crosslinker. In some embodiments, the reversible crosslinker is N,N′-Bis(acryloyl)cystamine (BAC) gel.

In some embodiments, the BAC is present at a concentration of about 0.150% to about 0.400%. In some embodiments, the BAC is present at about 0.200%.

In some embodiments, the gel floor layer is approximately 50 μm in height. In some embodiments, the second microwell comprises an elastomer. In some embodiments, the elastomer is selected from the group consisting of PDMS and a curing agent (10:1).

In some embodiments, the second microwell comprises PDMS and is approximately 200 μm in height and comprises microwells approximately 250 μm by 350 μm.

In some embodiments, the apparatus further comprises a glass slide comprising a plurality of through holes, the glass slide positioned between the first microwells and the second microwells, each through hole aligned with one of the first microwells and one of the second microwells.

In some embodiments, the apparatus further comprises a vacuum manifold layer coupled to the second microwells, the vacuum manifold layer comprising a plurality of pillars surrounding each of the second microwells, wherein a vacuum force is to be applied to the vacuum manifold layer to urge the organelles to move from the first microwells and to be collected in the individual second microwells.

In some embodiments, the second microwells each comprise a second microwell floor layer, wherein an interaction between the pillars and the second microwell floor layer enables substantially continued contact between the glass slide and the second microwells, and prevention of microwell deformation when the vacuum force is applied.

In some embodiments, the continued contact between the glass slide and the microwell floor layer reduces deformation of the second microwell floor layer in response to the vacuum force being applied.

In some embodiments, the glass slide has a thickness of about 400-μm.

In some embodiments, the apparatus further comprises a vacuum manifold layer coupled to the second microwells, wherein a vacuum force is to be applied to the vacuum manifold layer to urge the organelles to move from the first microwells and to be collected in the individual second microwells.

In some embodiments, the second microwells comprise a second microwell floor layer that is responsive to the vacuum force.

In some embodiments, the second microwell floor layer is to move in response to the vacuum force being applied to draw the reducing agent to the first microwell, dissolve the gel floor layer, and drive the organelles to move from the first microwells and to be collected in the individual second microwells.

In some embodiments, the vacuum manifold layer comprises one or more vacuum outlets to enable the vacuum force to be created within the vacuum manifold layer.

In some embodiments, the vacuum manifold layer comprises a plurality of pillars surrounding each of the second microwells. In some embodiments, the pillars comprise a trapezoidal cross-section. In some embodiments, the pillars deter the second microwells from collapsing or deforming when the vacuum force is applied to the second microwells.

In some embodiments, the apparatus further comprises barcoded beads in the second microwells.

In some embodiments, the barcoded beads and the second microwells comprise oligonucleotide primers capable of hybridizing to nucleic acids of the organelles. In some embodiments, the second microwells are loaded with barcodes.

### INCORPORATION BY REFERENCE

All publications, patents, and patent applications mentioned in this specification are herein incorporated by reference for all purposes and to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A depicts a schematic of an exemplary method of isolating biomolecules and organelles from a single cell.

FIG. 1B depicts a chart of the exemplary method of isolating biomolecules and organelles from a single cell of FIG. 1A.

FIG. 1C depicts a schematic of detailed isometric cross-sectional view of an example multilayer microfluidic apparatus of the disclosure.

FIG. 1D depicts the schematic of detailed isometric cross-sectional view of an example multilayer microfluidic apparatus with a vacuum force applied.

FIG. 1E depicts a representative image of an isometric bottom view of the multilayer microfluidic apparatus showing the vacuum manifold layer.

FIG. 1F depicts a representative image of a detailed view of the vacuum manifold layer showing the pillars surrounding one of the second microwells.

FIG. 2A depicts a schematic of a method for isolating biomolecules from a single cell, using the apparatus of the disclosure.

FIG. 2B depicts a schematic of the synthesis and dissolution mechanism of the dissolvable polyacrylamide BAC-gel used to create and open the trapdoor feature.

FIG. 2C depicts a schematic of an exploded view of the Bis-gel, the BAC-gel, the through-hole glass slide, and the PDMS microwell.

FIG. 3A depicts a schematic of an exemplary method of fabricating the composite gel comprising the Bis gel, the BAC-gel, and the second microwells.

FIG. 3B-3C depict schematics of an exemplary method of barcoding the second microwells.

FIG. 4A depicts the schematic of FIG. 2C.

FIG. 4B depicts a representative brightfield microscope image of the Bis gel microwell with the BAC gel layer aligned with the PDMS microwell.

FIG. 4C depicts a representative microscope image of the Bis gel layer having different heights or diameters.

FIG. 5A depicts a representative image of the Big-gel layered on top of the BAC-gel.

FIG. 5B depicts images of the microwell under different UV-dose ranges under wet conditions or dry conditions.

FIG. 5C depicts a graph of the measured diameter of the microwell under the different UV-dose ranges under wet or dry conditions.

FIG. 6A depicts a representative image of the microwell comprising trapezoid pillars with no vacuum force (left) and with vacuum force (right).

FIG. 6B depicts a representative image of the microwell with circular pillars under no vacuum force (left) and with vacuum force (right).

FIG. 6C depicts representative images of a time lapse of the microwell under vacuum force (top) and with no vacuum force (bottom)

FIG. 6D depicts representative images of the BAC gel under vacuum force (left, top and bottom) and the BAC gel under no vacuum force (right, top and bottom).

FIG. 6E depicts a graph of the percent of dissolution with and without the vacuum force.

FIGS. 7A-7C depict graphs of the dissolution rate, including BAC concentration (FIG. 7A), DTT concentration (FIG. 7B), and UV dose (FIG. 7C) used in Bis-gel photopolymerization.

FIG. 7D depicts a representative image of the Bis-gel, BAC-gel and an overlay of the Bis-gel and BAC-gel before dissolution (top) and after dissolution (bottom).

FIGS. 8A-8B depict representative images of a time lapse (FIG. 8A) or fluorescence images (top, middle, bottom of FIG. 8B) of nucleus transfer through the trapdoor of the BAC-gel into the nuclei-receiving PDMS microwell.

FIGS. 8C-8D depict graphs of the percentage of wells that had BAC-gel dissolution, successful nucleit transfer or misalignment inspected by microscopy. during BAC-gel dissolution inspection of the microwells by microscopy during BAC-gel dissolution revealed that ~80% of the BAC-gel microwells dissolved, corresponding with the percentage of nuclei transferred within the same microwell array

FIG. 8E depicts representative images of different magnification of nuclei after transfer.

FIG. 8F depicts representative images of nuclei before and after transfer.

FIG. 8G depicts graphs of common morphological parameters measured of nuclei before and after transfer.

FIG. 9A depicts representative micrographs showing bead microwell filling.

FIGS. 9B-9C depicts graphs of and representative images of microwell filling for side dimensions of 200 μm (FIG. 10B) and 300 μm (FIG. 10C) and several plasma treatment conditions.

FIG. 9D depicts a graph of the well-to-well uniformity of fluorescently labeled bead signal averaged across a microwell based on flow rate of liquid containing beads.

FIGS. 9E-9F depict deposition patterns observed after microwell drying (FIG. 10E) and a SEM image for bead distribution between microwells with bottom surface sedimentation and microwells with only bead-wall attachment (FIG. 10F).

FIG. 10A depicts a schematic of a COMSOL simulation of balance of forces on the bead.

FIG. 10B depicts a representative micrograph of the microwells after flow was passed through in an orthogonal direction at different flow rates.

FIG. 11A depicts a snATAC-seq electropherogram profile for 256 uniquely barcoded microwell deposited by 2.5/mL barcoded magnetic beads and 10 μM free oligos.

FIG. 11B depicts a representative image of 2 MCF 7 nuclei inside a microwell at 4× and 40×.

FIG. 12A-12D depicts an exemplary, complete assay using MCF7 cells.

### DETAILED DESCRIPTION OF THE INVENTION

It is to be understood that this application is not limited to particular formulations or process parameters, as these may, of course, vary. It is also to be understood that the terminology used herein is for the purpose of describing particular embodiments only and is not intended to be limiting. Further, it is understood that a number of methods and materials similar or equivalent to those described herein can be used in the practice of the present disclosure.

In accordance with the present application, there may be employed conventional molecular biology, microbiology, and recombinant DNA techniques as explained fully in the art. The definitions contained herein supplement those in the art and are directed to the current application and are not to be imputed to any related or unrelated case, e.g., to any commonly owned patent or application. Accordingly, the terminology used herein is for the purpose of describing particular embodiments only and is not intended to be limiting.

In this application, the use of the singular includes the plural unless specifically stated otherwise. It must be noted that, as used in the specification, the singular forms “a,” “an” and “the” include plural referents unless the context clearly dictates otherwise. Furthermore, use of the term “including” as well as other forms, such as “include”, “includes,” and “included,” is not limiting.

The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.”

The term “or” can be used conjunctively or disjunctively, unless the context specifically refers to a disjunctive use.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the disclosure can be used to achieve methods of the disclosure.

As used herein the term “consisting essentially of” refers to those elements required for a given embodiment. The term permits the presence of elements that do not materially affect the basic and novel or functional characteristic(s) of that embodiment of the disclosure.

As used herein the term “consisting of” refers to compositions, methods, and respective components thereof as described herein, which are exclusive of any element not recited in that description of the embodiment.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosure.

### DETAILED DESCRIPTION

Although the following text discloses a detailed description of implementations of methods, apparatuses and/or articles of manufacture, it should be understood that the legal scope of the property right is defined by the words of the claims set forth at the end of this patent. Accordingly, the following detailed description is to be construed as examples only and does not describe every possible implementation, as describing every possible implementation would be impractical, if not impossible. Numerous alternative implementations could be implemented, using either current technology or technology developed after the filing date of this patent. It is envisioned that such alternative implementations would still fall within the scope of the claims.

### Multi-Modal, Same Cell Assay

With a focus on introducing tools for single-cell resolution protein measurement, this disclosure introduces a suite of single-cell immunoblotting modalities designed using microwell-isolated single cells, including single-cell western blots [29-31]. With an eye towards organelle-biology and subcellular omics, this application includes tools for single-nucleus isolation using microwell-isolated mammalian cells subjected to differential detergent fractionation (DDF), a technique that employs a sequence of detergents with varying solubilization strengths to selectively extract and separate cellular components based on membrane properties [2, 32]. In one example of a single-nucleus resolution analysis that is made possible by combining microfluidic precision with DDF, a single-cell Western blot of each cytoplasmic compartment and a distinct electrophoresis of each nuclear compartment for an array of cells may be performed. In a second example that extends on the assay just described, the Western blot analysis of each single nucleus may be swapped out with a PCR assay, allowing both cytoplasmic protein targets and nuclear DNA and RNA targets to be detected in the same originating cell [33-35]. While both single-nucleus precision assays are suitable for sparingly available starting specimens (<10 starting cells, e.g., isolated circulating tumor cells, individual blastomeres comprising two-and four-cell preimplantation murine embryos), sample and analysis throughput must be increased for applicability to larger-cell-number specimens.

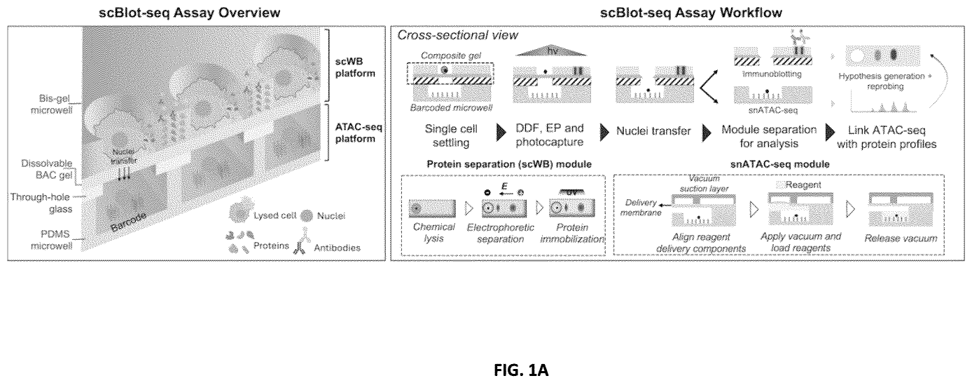

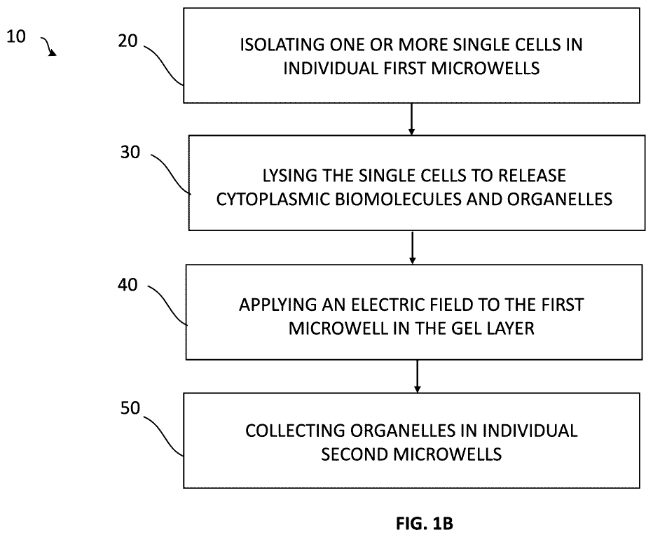

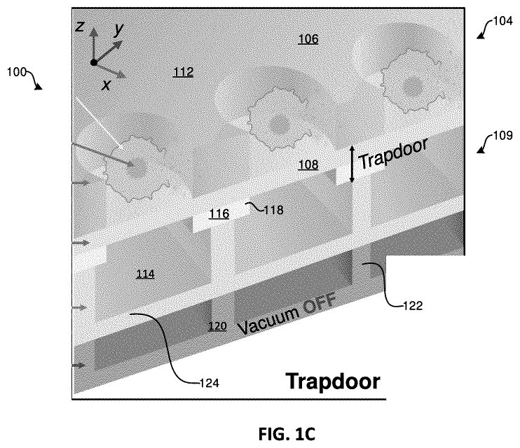

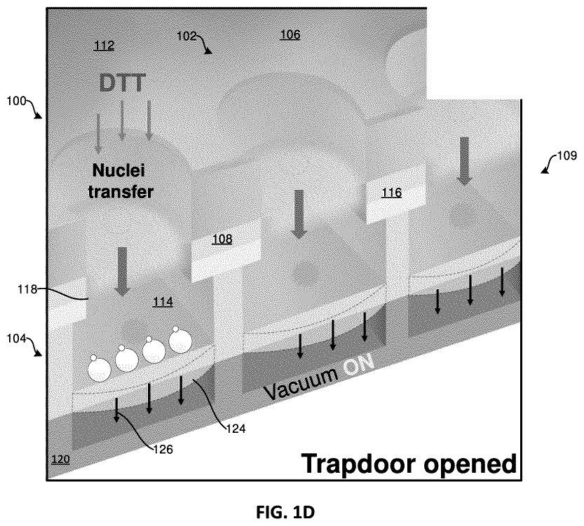

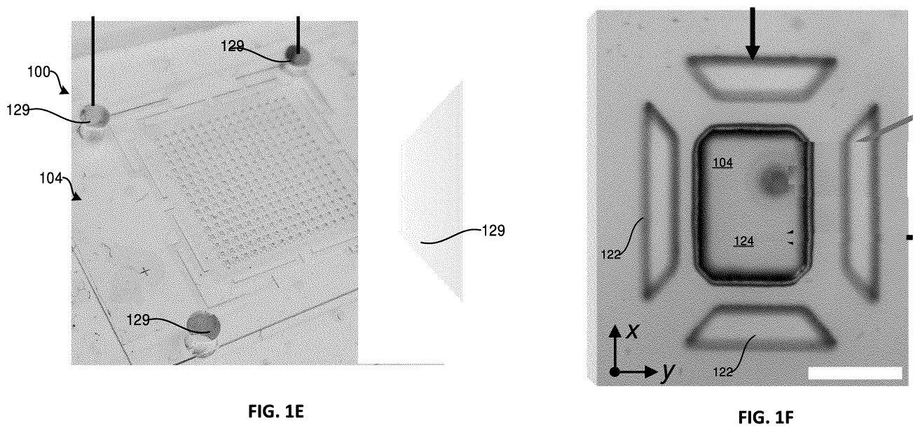

FIG. 1A depicts a schematic of an exemplary method of an exemplary method of isolating biomolecules and organelles from a single cell. For example, scBlot-seq combines measurements of chromatin accessibility via snATAC-seq and proteomics via single-cell Western blot (scWB) (PCT/US24/24096, incorporated by reference in its entirety herein) from the same single cell. This method addresses the limitations of current multimodal single-assay methods by enabling isoform detection, eliminating the need for prior knowledge of protein targets or cellular fixation, and establishing a target-discovery-target analysis within the same cell. Initially, single cells may be captured within the composite polyacrylamide microwells (Bis-gel). Subsequent steps may involve scWB, including partial cell lysis by differential detergent fractionation (DDF), electrophoresis (EP), and photocapture, which may lead to the immobilization of proteins within the gel while initiating the transfer of nuclei by dissolving the BAC gel layer. The scWB gel may then be archived for later analysis, and the snATAC-seq platform may undergo preparation for on-chip library preparation. Upon sequencing and analysis of the snATAC-seq data, the chromatin accessibility profiles facilitate the identification of protein targets, while iterative gel reprobing by immunoprobing for proteins of interest enables a target-discovery-target assay within the same cell. This apparatus and method maintain the spatial link between isolated organelles and their originating cells or protein content, allowing for more precise single-cell multiomics analysis. By maintaining the connection between organelles and their originating cells, this apparatus and method is ideal for applications requiring multimodal, single-cell analyses.

A single-cell resolution organelle isolation method is introduced incorporating a single-cell isolation via a polyacrylamide microwell array that is optimized for nuclear isolation after DDF. Microfluidic automation is utilized to enhance throughput while offering the capacity to isolate and then index individual nuclei back to each originating cell. In a multi-layered, planar microfluidic device, individual cells are isolated by settling into an array of polyacrylamide microwells, one cell per microwell. Perturbation, proteomics, or imaging analysis can be performed on intact cells in these top-layer polyacrylamide gel microwells. To isolate and extract single nuclei for further analysis, each cell's cytoplasmic membrane is lysed using DDF, and one intact nucleus remains in each microwell. To concurrently transfer each nucleus to an aligned PDMS microwell situated below the PAG microwell, an interleaving layer of through holes filled with a dissolvable gel is actuated. The dissolvable ‘trapdoor’ in the floor of each PAG microwell opens when the dissolvable gel is exposed to reducing agents (i.e., dithiothreitol (DTT)) and suction is applied using an attached microfluidic vacuum manifold. Once the trapdoors are open, hundreds of nuclei are simultaneously transferred from the PAG microwell array to the PDMS microwell array, at one nucleus per microwell occupancy. The multi-layer fluidic design, chemical and hydrodynamic control optimization, and resultant organelle isolation and extraction performance of this single-nucleus isolation and extraction technique are detailed herein.

### Methods for Isolating Biomolecules and Organelles From a Single Cell

Disclosed herein is a method for isolating biomolecules and organelles from a single cell in separate chambers. In some embodiments, the biomolecules may include cytoplasmic (e.g., PI3K, AKT, mTOR, PTEN, STAT, or the like) and/or membrane proteins (e.g. HER2, EGFR, EpCAM, CD44, or the like). It can be appreciated that other biomolecules may be isolated using the methods described below. In some embodiments, the organelles may comprise nuclei, the endoplasmic reticulum, mitochondria, and ribosomes. It can be appreciated that other cellular organelles may be isolated using the methods described below.

In some embodiments, the method 10 (as shown in the schematic of FIG. 1B) comprises: isolating one or more single cells in individual first microwells 20, wherein said first microwell comprises a gel layer and a gel floor layer; lysing the one or more single cells to release cytoplasmic biomolecules and organelles 30; applying an electric field to the first microwell in the gel layer, thereby separating cytoplasmic biomolecules within the gel layer 40; collecting organelles in individual second microwells for further analysis 50; thereby isolating cytoplasmic biomolecules and organelles from a single cell in separate chambers.

In some embodiments, the method for isolating biomolecules and organelles from a single cell in separate chambers may also be used to as a method for sample preparation for future or further analysis of biomolecules and organelles from a single cell and can comprise the same and/or similar steps.

In some embodiments, isolating one or more individual cells in individual first microwells comprise depositing one more single cells into individual microwells from a microfluidic channel. In some embodiments, isolating one or more individual cells may comprise isolating one or more individual cells by flow cytometry and cell sorting. In some embodiments, depositing one or more single cells into individual microwells may be done via gravitational settling. In some embodiments, isolating one or more individual cells may comprise isolating about 6, about 12, about 24, about 48, about 96, about 258, about 384 or about 1536 single cells and depositing each into individual microwells. In some embodiments, the one or more individual cells may be selected form the group consisting of mammalian cells, human cells, tumor cells, organoids, patient-derived cells, patient-derived organoids, and dissociated human tissues. It can be appreciated that other types of cells may be isolated and deposited into individual microwells. In some embodiments, isolating one or more individual cells may comprise using a stacked microwell pair comprising a first microwell and a second microwell, as will be described in more detail herein. In some embodiments, isolating one or more single cells may comprise using an apparatus to isolate the one or more single cells, and the apparatus will be described in more detail herein.

In some embodiments, isolating one or more single cells in individual first microwells may comprise isolating concurrently or within a range of time of seconds or minutes. For examples, in some embodiments, isolating may comprise isolating within about 30 seconds, within about 60 seconds, within about 90 seconds, within about 150 seconds, within about 180 seconds, within about 200 seconds, within about 250 seconds, or within about 300 seconds. In some embodiments, isolating may comprise isolating within about 6 minutes, within about 7 minutes, within about 8 minutes, within about 9 minutes, or within about 10 minutes.

In some embodiments, the gel layer of the first microwell may comprise one or more of Bis/acrylamide monomer, a photoinitiator, a UV photoimmobilizer, a buffer, and water. In some embodiments, the photoinitiator comprises VA-086. In some embodiments, the UV photoimmobilizer comprises BPMA. In some embodiments, the buffer comprises Tris-HCl. In some embodiments, the Bis-acrylamide monomer comprises Acrylamide/Bis-acrylamide 40% solution (29:1). In some embodiments, the total percentage concentration of acrylamide monomer including the crosslinker (% T) may be any percentage and the percent of that total monomer made up of the crosslinker (% C) may be any percent. In some embodiments, the % T may be about 7% and the % C may be about 3.3%. It can be appreciated that the % T and % C may be changed or adjusted depending on the size of the target biomolecule.

In some embodiments, the gel layer may comprise a Bis-gel that is approximately 60 μm in height and may comprise microwells approximately 40 μm in diameter. It can be appreciated that the Bis-gel may comprise other dimensions for the height and the microwells of the Bis-gel may comprise other dimensions for the diameter, as will be described in more detail herein.

In some embodiments, the gel floor layer may comprise one or more of: a reversible crosslinker, acrylamide, TEMED, and APS. In some embodiments, the reversible crosslinker may be N,N′-Bis(acryloyl)cystamine (BAC) gel. In some embodiments, BAC may be present at a concentration of about 0.150% to 0.400%, including at about 0.200%, although other concentrations are contemplated as described in more detail herein.

In some embodiments, the gel floor layer may be approximately 50 μm in height. In some embodiments, the gel floor can be dissolved by contacting the gel floor with a reducing agent. Dissolving the gel floor may allow organelles to pass from the first microwell to the second microwell. It can be appreciated that the gel floor layer may comprise other dimensions as will be described in more detail herein.

In some embodiments, lysing the one or more single cells to release cytoplasmic biomolecules and organelles comprises contacting the one or more single cells with a chemical lysing agent. In some embodiments, the chemical lysing agent comprises a differential detergent fractionation (DDF) agent. In some embodiments, the DDF agent comprises a buffer comprising Digitonin, Triton-X, Brij-35, Igepal CA-630, CHAPS, n-dodecyl-b-D-maltopyranoside, or the like. In some embodiments, the buffer comprising the DDF agent lyses the cytoplasmic membranes but may leave the organellar membranes (e.g., organelles) intact.

In some embodiments, the electric field may be applied to the gel layer under conditions that allow for separation of the cytoplasmic biomolecules within the gel layer based on molecular weight (MW) and/or other chemical properties. In some embodiments, the electric field may be applied to the entire gel layer simultaneously to allow for separation of the cytoplasmic biomolecules within each individual first microwell. In some embodiments, the electric field may be applied across the gel layer for a specific time and with a specific intensity. For example, the electric field may be applied across the gel layer for a time range of about 1 second to about 60 seconds, including about 5 seconds, about 10 seconds, about 15 seconds, about 20 seconds, about 25 seconds, about 30 seconds, about 35 seconds, about 40 seconds, about 45 seconds, about 50 seconds, about 55 seconds, or about 60 seconds. In some embodiments, the electric field may be applied across the gel layer for longer than 60 seconds including for about 1 minute to about 5 minutes, including about 1 minute, about 2 minutes, about 3 minutes, about 4 minutes, or about 5 minutes.

In some embodiments, the second microwell confirmed for collecting the isolated organelles may comprise an elastomer. In some embodiments, the elastomer may be selected from the group consisting of PDMS and a curing agent (10:1). It can be appreciated that other elastomers can be considered to comprise the second microwell.

In some embodiments, the second microwell comprises PDMS and is approximately 200 um in height. In some embodiments, the second microwell may be approximately 250 μm in height by 350 μm in diameter.

In some embodiments, collecting organelles in individual second microwells includes dissolving the gel floor layer and applying a vacuum force to pass the organelles from the first microwells to the second microwells. In some embodiments, dissolving the gel floor layer comprises contracting the gel floor with a reducing agent. In some embodiments, the reducing agent is dithiothreitol (DTT). In some embodiments, dissolving the gel floor may be complete in approximate 3-5 minutes. It can be appreciated that other lengths of time are contemplated for dissolving the gel floor as may be described in more detail herein. The use of a dissolvable BAC gel layer combined with a vacuum-assisted transfer ensures minimal damage to organelles, preserving their structural and functional integrity. The simultaneous transfer of organelles from hundreds of cells within minutes with only a single step of reagent deposition (DTT) demonstrates a significant improvement throughput compared to previous methods.

In some embodiments, applying a vacuum force comprises applying a vacuum force with a vacuum manifold. In some embodiments, applying a vacuum force comprises applying a vacuum force of approximately 30-130 mbar. In some embodiments, applying a vacuum force comprises applying a vacuum force of about 30 mbar, about 40 mbar, about 50 mbar, about 60 mbar, about 70 mbar, about 80 mbar, about 90 mbar, about 100 mbar, about 110 mbar, about 120 mbar, or about 130 mbar. In some embodiments, applying the vacuum force does not cause the second microwell to collapse.

In some embodiments, the second microwell comprises barcoded beads. In some embodiments, the barcoded beads and the second microwells comprise oligonucleotide primers capable of hybridizing to nucleic acids of the organelles. In some embodiments, the second microwell may be loaded with barcodes. In some embodiments, the method for isolating biomolecules and organelles from a single cell may further comprise pooling the barcoded beads comprising hybridized nucleic acids for amplification and/or sequencing as will be described in more detail herein.

### Method for Detecting One or More Cytoplasmic Biomolecules and One or More Organelles From a Single Cell

Also disclosed herein is a method for detecting one or more cytoplasmic biomolecules and one or more organelles from a single cell. In some embodiments, the method comprises: isolating cytoplasmic biomolecules and organelles from a single cell, detecting one or more cytoplasmic biomolecules, and detecting one or more organelles, thereby detecting one or more cytoplasmic biomolecules and one or more organelles from a single cell.

In some embodiments, isolating cytoplasmic biomolecules and organelles from a single cell may include isolating cytoplasmic biomolecules and organelles as described above and in more detail herein.

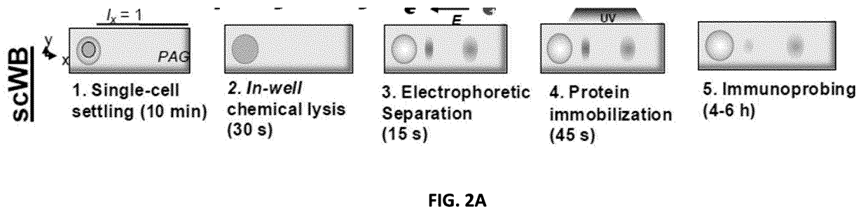

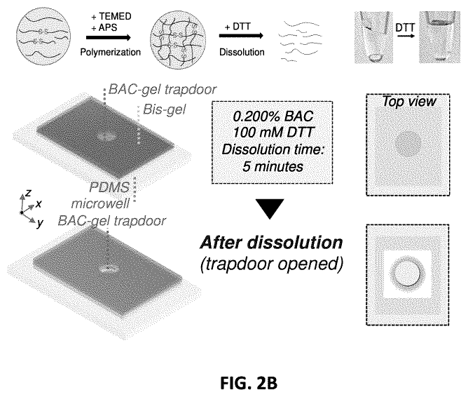

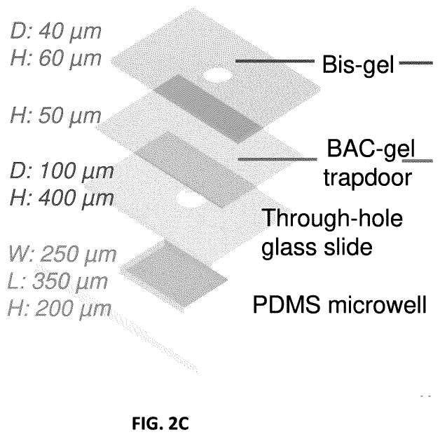

In some embodiments, detecting one or more cytoplasmic biomolecules comprises any one or more of detection methods comprising electrophoresis, immunoprobing and/or mass spectrometry, as seen in the schematic of FIG. 1A or FIG. 2A.

In some embodiments, detecting one or more organelles may comprise any one or more detection methods comprising sequencing, imaging, morphology identification, in situ hybridization and rolling circle amplification. In some embodiments, detecting the one or more organelles may comprise transferring the one or more organelles for the detection of information from nucleic acids (e.g., transcriptome, genome, chromatin accessibility, or the like). In some embodiments, detecting the one or more organelles may comprise transferring the one or more organelles for imaging.

In some embodiments, the cytoplasmic biomolecules may be covalently fixed within the gel layer prior to detecting or more may be transferred to a membrane prior to detecting.

In some embodiments, the organelles may be contracted in the second microwell by oligonucleotides corresponding to primer pair sequences under conditions that allow hybridization of the primer pair to nucleic acids of the organelles, thereby forming a complex, and wherein the complex is subjected to amplification and/or sequencing. In some embodiments, the sequencing may be selected from the group consisting of snATAC-seq, scATAC-seq, scRNA-seq, snRNA-seq, DNA methylation, DNA-sequencing, and whole genome sequencing.

In some embodiments, detecting of the one or more cytoplasmic biomolecules is linked to the one or more organelles from the same single cell.

In some embodiments, the method may further comprise a step of capturing one or more images of the cytoplasmic biomolecules and/or organelles. Capturing one or more images of the cytoplasmic biomolecules may comprise using any standard image capturing system, or any fluorescent imaging system.

Also disclosed herein is a method of linking information with protein information from a single cell. In some embodiments the method comprises: isolating cytoplasmic biomolecules and organelles from a single cell, detecting one or more cytoplasmic biomolecules, and detecting one or more organelles, thereby detecting one or more cytoplasmic biomolecules and one or more organelles from a single cell.

In some embodiments, isolating cytoplasmic biomolecules and organelles from a single cell may include isolating cytoplasmic biomolecules and organelles as described above and herein. In some embodiments, detecting one or more cytoplasmic biomolecules comprises any one or more of detection methods comprising electrophoresis, immunoprobing and/or mass spectrometry. In some embodiments, detecting one or more organelles may comprise any one or more detection methods comprising sequencing, imaging, morphology identification, in situ hybridization and rolling circle amplification.

Also disclosed herein is a method of united protein and nucleic acid-based omics analysis from the same cell. In some embodiments, the method comprises: isolating cytoplasmic biomolecules and organelles from a single cell, detecting one or more cytoplasmic biomolecules, and detecting one or more organelles, thereby detecting one or more cytoplasmic biomolecules and one or more organelles from a single cell.

In some embodiments, isolating cytoplasmic biomolecules and organelles from a single cell may include isolating cytoplasmic biomolecules and organelles as described above and herein. In some embodiments, detecting one or more cytoplasmic biomolecules comprises any one or more of detection methods comprising electrophoresis, immunoprobing and/or mass spectrometry. In some embodiments, detecting one or more organelles may comprise any one or more detection methods comprising sequencing, imaging, morphology identification, in situ hybridization and rolling circle amplification.

Also disclosed herein is a method of mapping isolated organelles to an original cell of origin (spatial indexing. In some embodiments, the method comprises isolating cytoplasmic biomolecules and organelles from a single cell, detecting one or more cytoplasmic biomolecules, and detecting one or more organelles, thereby detecting one or more cytoplasmic biomolecules and one or more organelles from a single cell.

In some embodiments, isolating cytoplasmic biomolecules and organelles from a single cell may include isolating cytoplasmic biomolecules and organelles as described above and herein. In some embodiments, detecting one or more cytoplasmic biomolecules comprises any one or more of detection methods comprising electrophoresis, immunoprobing and/or mass spectrometry. In some embodiments, detecting one or more organelles may comprise any one or more detection methods comprising sequencing, imaging, morphology identification, in situ hybridization and rolling circle amplification.

### Apparatuses

Also disclosed herein are apparatuses that may be configured for isolating and separating cytoplasmic biomolecules and organelles. In some embodiments, an apparatus may comprises: a plurality of first microwells, wherein the first microwells comprise a gel layer and a gel floor layer; a plurality of second microwells, each first microwell aligned with one of the second microwells, wherein one or more single cells are to be lysed within the first microwells to release cytoplasmic biomolecules and organelles, wherein an electric field is to be applied to the first microwell in the gel layer, thereby separating cytoplasmic biomolecules within the gel layer, and wherein the gel floor layer is to be dissolved to enable organelles to be collected in the individual second microwells.

FIG. 1C is a detailed isometric cross-sectional view of an example multilayer microfluidic apparatus 100 in accordance with the teaching of this disclosure. The multilayer microfluidic device 100 includes a plurality of first microwells 102 and a plurality of second microwells 104. The first microwells 102 include a gel layer 106 and a gel floor layer 108. Each of the first microwells 102 is aligned with one of the second microwells 104 in the implementation shown. The first microwells 102 that are aligned with a corresponding one of the second microwells 104 form a stacked microwell pair 109.

One or more single cells 110 may be lysed within the first microwells 102 in operation to release cytoplasmic biomolecules and organelles. An electric field may be applied to the first microwell 102 in the gel layer 106, thereby separating cytoplasmic biomolecules within the gel layer 106 and the gel floor layer 108 is dissolved to enable organelles to be collected in the individual second microwells 104. The gel floor layer 108 may be dissolved by contacting the gel floor with a reducing agent as an example.

In some embodiments, each of the first microwells may be aligned with one of the second microwells to form a stacked microwell pair. In some embodiments, the apparatus may further comprise a plurality of stacked microwell pairs, each microwell pair comprising one of the first microwells and one of the second microwells. In some embodiments, the plurality of stacked wells may be arranged in any configuration. In some embodiments, the plurality of wells may comprise an even number of wells. For example, in some embodiments, the plurality of wells may comprise about 6 wells, about 8 wells, about 12 wells, about 16 wells, about 24 wells, about 32 wells, about 48 wells, about 96 wells, about 132 wells, about 144 wells, about 256 wells, about 384 wells, or about 1536 wells.

In some embodiments, the gel floor is configured to be dissolved by contracting the gel floor with a reducing agent. The gel layer 106 may be approximately 60 μm in height and include the first microwells 102 that are approximately 40 μm in diameter. The first microwells 102 may have a diameter larger than 40 μm or less than 40 μm. The gel layer 106 may include polyacrylamide in some implementations. The gel layer 106 may include a Bis gel 112 including acrylamide and bis-acrylamide in some implementations. The Bis gel 112 may include Acrylamide/Bis-acrylamide 40% solution (29:1).

In some embodiments, the gel layer may be approximately from about 10 μm to about 100 μm in height, including about 20 μm, about 30 μm, about 40 μm, about 50 μm, about 60 μm, about 70 μm, about 80 μm, about 90 μm, or about 100 μm in height. In some embodiments, the gel layer is approximately 60 μm in height.

In some embodiments, the gel layer may comprise microwells having diameters of about 10 μm to about 100 μm in diameter. In some embodiments, each microwell may have a diameter of about 10 μm, about 20 μm, about 30 μm, about 40 μm, about 50 μm, about 60 μm, about 70 μm, about 80 μm, about 90 μm, or about 100 μm in diameter. In some embodiments, each microwell has a diameter of about 40 μm.

It can be appreciated that the height and diameter of the microwells may be different to accommodate different cells. For example, for cells that are larger in size or diameter, the microwells may have an increased diameter and a greater height. For cells that are smaller in size or diameter, the microwells may have a comparatively smaller diameter and smaller height.

In some embodiments, each microwell may have a ratio of diameter to height of the gel layer of about 2:3. In some embodiments, each well may have a ratio of diameter to height of the gel layer of about 1:1, 1:2, 1:3, 1:4, about 1:5, about 1:6, about 1:7, about 1:8, about 1:10, about 2:5, about 3:4, about 5:8, or about 7:8.

In some embodiments, the gel layer may comprise one or more of Bis/acrylamide monomer, a photoinitiator, a UV photoimmobilizer, a buffer, and water. In some embodiments, the photoinitiator is VA-086 or LAP, the UV photoimmobilizer is BPMA, and the buffer comprises Tris-HCl.

In some embodiments, the gel layer comprises a Bis gel comprising acrylamide and bis-acrylamide. In some embodiments, the Bis gel comprises Acrylamide/Bis-acrylamide 40% solution (29:1). It can be appreciated that other ranges of acrylamide/Bis-acrylamide solution can be used including about 10%, about 15%, about 20%, about 25%, about 30%, about 35%, about 40%, about 45%, or about 50%. In some embodiments, the Bis gel comprises Acrylamide/Bis-acrylamide solution of about 19:1, 29:1, or 37.5:1.

In some embodiments, the Bis gel comprises acrylamide/Bis-acrylamide of about 1% to about 15%. In some embodiments, the Bis gel comprises acrylamide/Bis-acrylamide of about 4% to about 12%. In some embodiments, the Bis gel comprises acrylamide/Bis-acrylamide of about 7% to about 8%. It can be appreciated that in some embodiments, the Bis gel may comprise acrylamide/Bis-acrylamide of any %

In some embodiments, the gel floor layer comprises a reversible cross linker. The gel floor layer may be dissolvable to transfer material from the gel layer of the first microwell to the second microwell of the microwell array. In some embodiments, the reversible cross linker may comprise N,N′-Bis(acryloyl)cystamine (BAC) gel. In some embodiments, the BAC may be present at a concentration of about 0.150% to about 0.400% about 0.1500%, about 0.200%, about 0.2500%, about 0.300%, about 0.3500%, or about 0.400%. In some embodiments, the BAC is present at about 0.200%. Other reversible crosslinkers may prove suitable, however.

In some embodiments, the reversible crosslinker may be dissolved via a chemical reaction, a mechanical application, or a combination thereof. For example, the reversible crosslinker may be dissolved by the application of a reducing agent. In some embodiments, the reducing agent may comprise dithiothreitol (DTT), although other reducing agents are contemplated. In some embodiments, the mechanical application may comprise the application of a vacuum force to the reversible crosslinker.

In some embodiments, the gel floor layer 108 may be approximately 50 μm in height. The gel floor layer 108 may have a height greater than 50 μm or a height less than 50 μm, however. For example, in some embodiments, the gel floor layer may comprise a height from about 10 μm to about 100 μm. In some embodiments, the gel floor layer may comprise a height of about 10 μm, about 20 μm, about 30 μm, about 40 μm, about 50 μm, about 60 μm, about 70 μm, about 80 μm, about 90 μm, or about 100 μm.

In some embodiments, the second microwells 104 may include an elastomer 114. The elastomer 114 is selected from the group consisting of PDMS, and/or a curing agent (10:1). The elastomer 114 may include and/or be implemented by other materials, however.

In some embodiments, the second microwell comprises PDMS and is approximately 200 um in height. The second microwells 104 may be a different dimension, however. In some embodiments, the second microwell may comprise a height from about 100 μm to about 300 μm. In some embodiments, each of the second microwells may comprise a height of about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, or about 300 μm. In some embodiments, each of the second microwell may comprise a height of 250 μm.

In some embodiments, the second microwell has a diameter of approximately 350 μm. The second microwell 104 may have a different diameter. For example, the second microwells may comprise a diameter of about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380 μm, about 390 μm, or about 400 μm. In some embodiments, the second microwell has dimensions of approximately 250 μm by 350 μm.

In some embodiments, the apparatus may comprise a vacuum manifold layer coupled to the second microwells, the vacuum manifold layer comprising a plurality of pillars surrounding each of the second microwells, wherein a vacuum force is to be applied to the vacuum manifold layer to urge the organelles to move from the first microwells and to be collected in the individual second microwells. In some embodiments, the vacuum manifold layer comprises one or more vacuum outlets to enable the vacuum force to be created within the vacuum manifold layer. Advantageously, the vacuum manifold layer in combination with the vacuum force and the reversible crosslinker enables for the simultaneous transfer or organelles from the first microwell to the second microwell within minutes using only a single step of reagent deposition (DTT) as described above and the application of the vacuum force.

The multilayer microfluidic device 100 of FIG. 1D is shown also including the vacuum manifold layer 120 coupled to the second microwells 104. The vacuum manifold layer 120 includes the plurality of pillars 122 surrounding each of the second microwells 104. A vacuum force is applied to the vacuum manifold layer 120 in operation to urge the organelles to move from the first microwells and be collected in the individual second microwells 104. Advantageously, this apparatus achieves an organelle transfer efficiency of up to 98%.

The second microwells 104 each include a second microwell floor layer 124. An interaction between the pillars 122 and the second microwell floor layer 124 may enable substantially continued contact between the glass slide 116 and the second microwells 104. The continued contact between the glass slide 116 and the second microwell floor layer 124 may reduce deformation of the second microwell floor layer 124 in response to the vacuum force being applied in some implementations.

In some embodiments, the second microwell floor layer may be ultra-thin. In some embodiments, the second microwell floor layer may be configured to move in response to the vacuum force being applied to draw the reducing agent to the first microwell, dissolve the gel floor layer, and drive the organelles to move from the first microwells and to be collected in the individual second microwells.

FIG. 1D is the detailed isometric cross-sectional view of the multilayer microfluidic device 100 showing the gel floor layer 106 dissolved, the second microwell floor layer 124 being responsive to the vacuum force, and the organelles being collected in the second microwells 104. For example, the second microwell floor layer 124 is shown moving in a direction generally indicated by arrows 126 in response to the vacuum force being applied to the vacuum manifold layer 120. The vacuum force may draw the organelles to move from the first microwells 102 and be collected in the individual second microwells 104 as shown.

In some embodiments, the apparatus may further comprise beads in the second microwell. In some embodiments, the beads may comprise barcoded beads and non-barcoded beads. In some embodiments, the barcoded beads and the second microwells may comprise oligonucleotide primers capable of hybridizing to nucleic acids of the organelles, as will be described in more detail herein.

Barcoded beads 127 are shown positioned in the second microwells 104. The barcoded beads 127 may be omitted in other implementations. The barcoded beads 127 and/or the second microwells 104 may include oligonucleotide primers 129 capable of hybridizing to nucleic acids of the organelles. The second microwells 104 may be loaded with barcodes in some examples. The barcodes may be the oligonucleotide primers 129.

FIG. 1E is an isometric bottom view of the multilayer microfluidic device 100 showing the vacuum manifold layer 120. The vacuum manifold layer 120 includes one or more vacuum outlets 129 that enable the vacuum force to be created within the vacuum manifold layer 120. The vacuum outlets 129 are shown positioned in the corners of the vacuum manifold layer 120. Four of the vacuum outlets 129 are shown. The vacuum manifold layer 120 may include more than four of the vacuum outlets 129 or less of the vacuum outlets 129 in other configurations.

FIG. 1F is a detailed view of the vacuum manifold layer 120 showing the pillars 122 surrounding one of the second microwells 104. The pillars 122 may surround each of the second microwells 104. The pillars 122 include a trapezoidal cross-section in the implementation shown. The pillars 122 may have a different cross-section, however. The pillars 122 may have a rectangular cross-section, an oblong cross-section, and/or a circular cross-section as examples. The pillars 122 may deter the second microwells 104 from collapsing or deforming in practice when the vacuum force is applied to the second microwells 104.

Also described herein is an apparatus comprising a plurality of microwells, wherein the first microwells comprise a gel layer and a gel floor layer, wherein one or more single cells are to be lysed within the microwells to release cytoplasmic biomolecules and organelles, wherein an electric field is to be applied to the microwell in the gel layer, thereby separating the cytoplasmic biomolecules within the gel layer, and wherein the gel floor layer is to be dissolved to enable the organelles to pass there through.

Also described herein is an apparatus comprising: a plurality of microwells; and a vacuum manifold layer coupled to the microwells, the vacuum manifold layer comprising a plurality of pillars surrounding each of the second microwells, wherein a vacuum force is to be applied to the vacuum manifold layer to urge organelles to be collected in the individual microwells.

### Method of Fabricating One or More Components of the Apparatus

Also disclosed herein are methods for fabricating one or more components of the apparatus including any one or more of the gel layer, the second microwell, and/or the vacuum manifold layer. Referring back to FIG. 1B, the multilayer microfluidic apparatus 100 of FIG. 1B is shown also including a glass slide 116 including a plurality of through holes 118. As seen in the schematic of FIG. 2C, the glass slide 116 is positioned between the first microwells 102 and the second microwells 104 and each through hole 118 is aligned with one of the first microwells 102 and one of the second microwells 104. The glass slide 116 may have a thickness of about 400-μm. The glass slide 116 may have another thickness, however.

In some embodiments, the first microwell may be polymerized on top of the glass slide. In some embodiments, the second microwell may be reversible bonded to the glass.

FIG. 2B depicts a schematic of the synthesis and dissolution mechanism of the dissolvable polyacrylamide BAC-gel used to create and open the trapdoor feature. Acrylamide monomers and BAC crosslinker undergo polymerization via C═C double bonds facilitated by ammonium persulfate (APS) and tetramethylethylenediamine (TEMED), resulting in the formation of a dissolvable polyacrylamide gel layer. Exposure to reducing agents (DTT) depolymerizes the disulfide-crosslinked BAC-gel due to the thiol-disulfide exchange reaction. Upon dissolution with DTT and application of a suction force, the through hole is opened and materials transfer from the top Bis-gel microwell into the bottom receiving PDMS microwell.

The mechano-chemically actuated trapdoor comprises a layer of reversibly crosslinked BAC hydrogel on a glass slide engraved with precision-drilled 100-μm diameter through holes, each aligned to a stacked microwell pair.

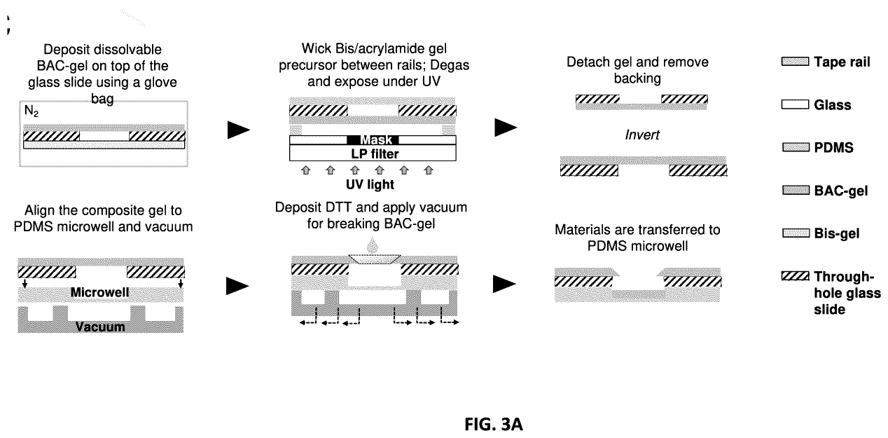

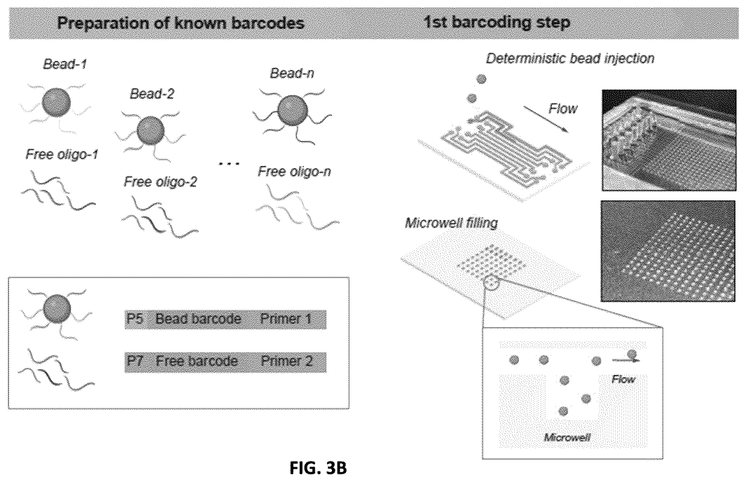

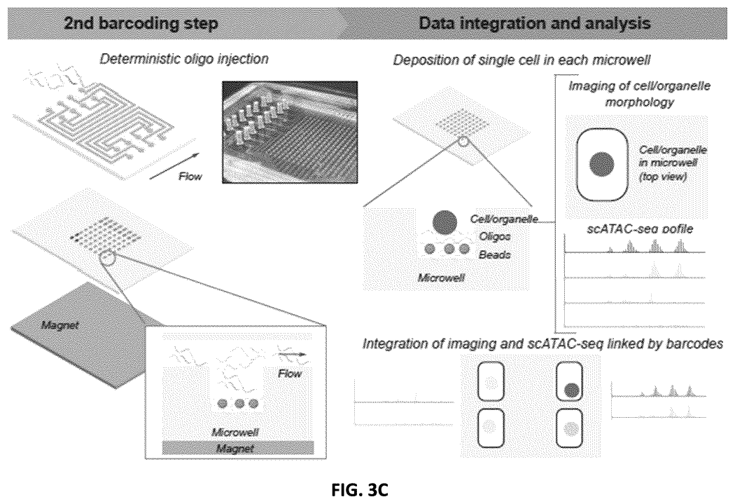

In some embodiments, the BAC-gel may be first polymerized atop a through-hole glass slide, creating a stable, covalently bonded trapdoor, as seen in FIG. 3A. In some embodiments, after the BAC-gel polymerizes, the Bis-gel is then polymerized directly on top of the BAC-gel, forming the microwells with the BAC-gel as a base of the microwell. This layered assembly allows for efficient isolation of intact mammalian cells for downstream applications, such as perturbation, imaging, or proteomics analysis. The through-hole glass slide may act as a gate for isolating organelles from each cell by providing structural support throughout the single-cell handling steps and ensuring stability during the alignment to the PDMS nucleus-receiving microwells (e.g., the second microwell). Without the support of the glass, the thin composite of BAC and Bis-gels (which in some embodiments may be approximately 100 um thick) would collapse during the dissolution process. Additionally, the through-hole glass slide forms a well-defined path for nuclei to travel from the Bis-gel microwells, through the trapdoor, and into the PDMS microwells. Advantageously, glass is an ideal material for this design due to its strong bonding properties with both polyacrylamide and PDMS.

For organelle transfer from the first microwell to the second microwell to be successful, the first microwell is polymerized and aligned atop the glass through hole that is coated with the BAC-gel. This will function as a trapdoor conduit receiving the second microwell. For example, the 40-μm diameter first microwell may be polymerized and aligned atop of each 100-μm diameter glass through hole coated with 50 μm-thick layer of the BAC-gel that will function as a trapdoor conduit to the receiving 250 by 350-μm PDMS microwell.

In some embodiments, the BAC-gel here may act as a temporary base of the first microwell to allow for alignment of each first microwell and second microwell across the entire array. To do this in a time efficient manner, the polymerization and formation process of the first microwell may be delayed until the location is defined to align with each of the through-hole of the glass slide.

To achieve this balance of timing, a distinct two polymerization methods for BAC-gel and Bis-gel may be implemented, wherein the BAC-gel and the Bis-gel may each have a different time constant for polymerization. For example, in some embodiments, the BAC-gel may be polymerized first and then the Bis-gel may be polymerized second. In some embodiments, the BAC-gel may be polymerized on top of the through-hole glass slide using acrylamide monomers and BAC as a cross-linker through free radical polymerization with tetramethylethylenediamine (TEMED) and Ammonium Persulfate (APS).

To precisely position the Bis-gel microwells directly over the BAC-gel-coated through holes—ensuring the effective transfer of nuclei across multiple layers of the VacTrap system—the Bis-gel may be photopolymerized using a photoinitiator. In some embodiments, the photoinitiator may comprise 2,2-Azobis[2-methyl-N-(2-hydroxyethyl)pro-pionamide] (VA-086, 1%). In some embodiments, a photomask may be employed to define the microwells diameter and location within the Bis-gel. In some embodiments, the Bis-gel may undergo UV exposure in order to photopolymerize the Bis-gel. For example, under UV exposure, the Bis-gel precursor in the transparent regions of the mask may be exposed and polymerized, while the opaque regions that be configured to define the shape and diameter of the microwells may block the UV light, preventing polymerization and forming the microwells. Advantageously, this photopolymerization approach allowed sufficient time to align the mask with the through holes, ensuring the Bis-gel microwells were accurately positioned over the BAC-gel-coated through holes, to form a composite gel.

To initiate the transfer of the gel to the PDMS microwells, the composite gel may be aligned to the PDMS microwell and the vacuum manifold. In some embodiments, this alignment may be done using brightfield microscopy.

As described above, the vacuum manifold layer comprises pillars having a trapezoid cross-sectional shape. The trapezoid pillar is configured to prevent the PDMS microwell from collapsing when a vacuum force is applied. This configuration may allow for continued contact between the through-hole glass slide and the PDMS microwell.

Additionally, the bifurcated channel network, having a channel height of approximate 100 μm, is fabricated on a separate thin membrane of PDMS (~300 μm) that is temporarily bonded to the microwell platform. This feature may be useful not only for library collection at the end but also for sensitive bioassays that require no contamination between reagents. In the automation systems, the sample spotting object needs to be decontaminated between each step of reagent loading which is time-consuming and not sufficient for time-sensitive assays. With this apparatus and method, the membrane can be removed within seconds and a new membrane can be attached for use within a few minutes.

### Method of Barcoding Microwells Suitable for Single Cell Analyses in a Microwell System

Also described herein are methods of barcoding microwells suitable for single cell analyses in a microwell system. Advantageously, coordinated barcoding using microfluidics may be achieved on genetic material that is deposited in the microwells after the barcoding, allowing for barcoding of free standing DNA, cells, microorganisms, and organoids.

While microfluidics could reduce the time and cost of the barcoding steps, current microfluidic technologies for barcoding are only compatible with patterned surfaces (e.g., tissues). To achieve deterministic coordinated barcoding of microwells, two chips with orthogonally located microfluidic channels may be used. In some embodiments, the first chip may be configured to deliver magnetic beads deterministically labeled with a barcode. After the bead has sedimented into the microwell, the first chip may be removed, and the second chip may be deposited with the channels orthogonally placed. In some embodiments, the second chip is held over a magnetic field during the injection of the second barcode. Free oligos are injected into the second chip, thus delivering a second set of barcodes into each microwell. After barcoding, the microwell array can be used for further analysis.

As seen in FIGS. 3B-3C, the method comprises labeling magnetic beads with known oligonucleotide barcodes, wherein the oligonucleotide barcodes comprise a bead barcode sequence and a first primer pair sequence; delivering the labeled magnetic beads into microwells using a microfluidic channel of a first microfluidic device; removing the first microfluidic device; applying a magnetic force under conditions that prevent the labeled magnetic beads from escaping the microwells; and delivering free oligonucleotides to the microwells comprising the labeled magnetic beads using a microfluidic channel of a second microfluidic device, wherein the free oligonucleotides comprise a free barcode sequence and a second primer pair sequence.

Advantageously, magnetic field may be used to prevent the cross-talk of barcodes coupled to the magnetic beads as they are delivered across the chip. In some embodiments, the magnetic beads may comprise an iron oxide core (e.g., Fe³O⁴. In some embodiments, the magnetic beads may comprise a diameter of about 1 μm to about 500 μm. In some embodiments, the magnetic beads may comprise a diameter of about 1 μm to about 100 μm, about 100 μm to about 200 μm, about 200 μm to about 300 μm, about 300 μm to about 400 μm, about 400 μm to about 500 μm.

In some embodiments, the magnetic beads may comprise a protective layer that may be modified by one or more chemical binding groups. In some embodiments, the protective layer may comprise dextran, although other compounds are contemplated.

In some embodiments, the magnetic beads may be coated with a chemical binding group configured to couple to the known oligonucleotide barcodes. In some embodiments, chemical binding group may comprise a streptavidin layer for a streptavidin-biotin binding of the known oligonucleotide barcodes. It can be appreciated that other chemical binding groups may be included including those configured for click chemistry or protein A/G binding.

As described above, a vacuum device may be configured to deliver barcoded beads to each well. In some embodiments, delivering the labeled magnetic beads into microwells using a microfluidic channel of a first microfluidic device includes delivering the labeled magnetic beads into the microwells in a first direction. In some embodiments, delivering the labeled magnetic beads into microwells may comprise delivering a minimum number of labeled magnetic beads into each microwell, delivering a maximum number of labeled magnetic beads into each microwell, delivering a minimum concentration of labeled magnetic beads into each microwell, or delivering a maximum concentration of labeled magnetic beads into each microwell. For example, in some embodiments, delivering the labeled magnetic beads into the microwell may comprise delivering about 5 mg/mL of labeled microbeads into each microwell.

In some embodiments, delivering free oligonucleotides to the microwells comprising the labeled magnetic beads using a microfluidic channel of a second microfluidic device includes delivering the free oligonucleotides to the microwell in a second direction perpendicular to the first direction. In some embodiments, the labeled magnetic beads are delivered into the microwell at a flow rate of about 1 μL/minute to about 1000 μL/minute. In some embodiments, the labeled magnetic beads are delivered into the microwell at a flow rate of about 1 μL/minute to about 100 μL/minute including about 1 μL/minute to about 10 μL/minute, about 10 μL/minute to about 20 μL/minute, about 20 μL/minute to about 30 μL/minute, about 30 μL/minute to about 40 μL/minute, about 40 μL/minute to about 50 μL/minute, about 50 μL/minute to about 60 μL/minute, about 60 μL/minute to about 70 μL/minute, about 70 μL/minute to about 80 μL/minute, about 80 μL/minute to about 90 μL/minute, or about 90 μL/minute to about 100 μL/minute.

In some embodiments, the first microfluidic device and the second microfluidic device may comprise microfluidic chips comprising orthogonal channels.

In some embodiments, the barcoded microwells comprise an elastomer including PDMS. In some embodiments, the second microwells comprise PDMS and are approximately 200 μm in height and comprise microwells approximately 250 μm by 350 μm.

This method of barcoding microwells can be achieved on genetic material that is deposited in the chip after the barcoding. This method can be used to barcode free standing DNA, cells, microorganisms, and organoids. Advantageously, using a magnetic field while delivering the magnetic beads prevents cross-talk of barcodes across the apparatus. By using plasma treatment of the barcoded surface but not chip layers, clamping is not necessary to prevent chip lifting. Additionally, this prevents permanent bonding, enabling the use of the microwells for downstream applications.

### Vacuum-Based Reagent Loading of the Apparatus

Also disclosed here is a method of highly parallel, dead-end, vacuum-based reagent delivery to microwells suitable for single cell analyses in a microwell system. Advantageously, the vacuum-based reagent delivery to the microwells may be configured so that reagents will arrive at each microwell at the same time through the bifurcated channel network. This simultaneous arrival of the reagents to each microwell may be extremely helpful for downstream time-sensitive applications (e.g., sequencing comprising a ATAC-seq: Tn5 tagmentation step). Also advantageously, the reagents may be delivered through the dead-end bifurcated channel network through both capillary force and vacuum power (both passive and active microfluidic device).

In some embodiments, the method comprises: bonding a bifurcated delivery membrane to a barcoded PDMS microwell, bonding a vacuum suction layer on top of the delivery membrane, and activating a vacuum force to generate negative pressure across the delivery membrane to drive the reagents to load each microwell simultaneously.

In some embodiments, bonding the bifurcated delivery membrane to the barcoded PDMS may be done before or after organelle transfer to the PDMS microwell.

In some embodiments, wherein the barcoded PDMS microwell is used for sequencing including PCR, mineral oil may be loaded into the microwell through the deliver membrane. In some embodiments, a self-cured oil mixture of 4:1500 cSt silicone oil with 10:3 PDMS mixture may be poured on top of the vacuum suction layer and a coverslip may be deposited on top of the entire system.

Advantageously, the apparatuses described herein and the methods of delivering reagents to microwells may not be limited to complete library preparation for sequencing; instead, these apparatuses and methods may be used for any applications that require different reagents loading at different certain time points. Examples of these applications may include but are not limited to whole/fractional cell lysis and digital PCR on chip, genomic DNA extraction by Ethanol Precipitation, or multimodal single cell analysis with protein analysis and nuclear mRNA sequencing.

### EXAMPLES

The following examples describe the methods and materials.

Bulk methods to fractionate organelles lack the resolution to capture single-cell heterogeneity. While microfluidic approaches attempt to fractionate organelles at the cellular level, they fail to map each organelle back to its cell of origin—crucial for multiomics applications. To address this, we developed VacTrap, a high-throughput microfluidic device for isolating and spatially indexing single nuclei from mammalian cells. VacTrap consists of three aligned layers: (1) a Bis-gel microwells layer with a ‘trapdoors’ (BAC-gel) base, fabricated atop a through-hole glass slide; (2) a PDMS microwell layer to receive transferred nuclei; and (3) a vacuum manifold. VacTrap operation begins with cell lysis using DDF to release intact nuclei into the Bis-gel microwells, while cytoplasmic proteins are electrophoresed into the Bis-gel. Upon exposure to DTT and vacuum force, the trapdoors open, allowing nuclei to transfer to the PDMS microwells. Vac Trap dissolves the trapdoors within 3-5 minutes and achieve synchronized nuclei transfer with 98% efficiency across 80% of trapdoors in a 256-microwell array, surpassing the <1% efficiency of passive transfer without vacuum. Morphology analysis confirmed preservation of organelle integrity throughout Vac Trap operation. By enabling spatial indexing of nuclei back to their original cell, VacTrap provides a robust, high-throughput tool for single-cell multiomics applications.

Cells are composed of specialized organelles that each perform unique functions. Structural and functional assays rely on organelle isolation, wherein the integrity of the isolated organelles directly affects analysis accuracy, ultimately shaping our understanding of biology [1]. Bulk organelle-fractionation methods (e.g., density-gradient centrifugation, immune-isolation, free-flow electrophoresis, detergent-based chemical fractionation, enzymatic digestion) are labor-intensive, designed for pooled cell suspensions and not suitable for sparingly available specimens, and offer low organelle-recovery yields. Although suffering from these performance shortcomings, density-gradient centrifugation remains widely used [2-4]. Immuno-isolation is constrained by the availability and quality of antibody probes specific to organelle surface proteins [5]. Free-flow electrophoresis separates cellular organelles [6, 7] with low recovery purity and resolution [8]. Detergent cocktails enrich specific cellular fractions; with each chemical component having a distinct solubilization efficiency [9, 10]. Yet, enzymatic treatments are known to perturb cell-cycle status, apoptosis, and structural alterations [11, 12]. Overall, bulk organelle-isolation methods require multiple steps requiring extensive manual handling and yield compromised purity and integrity of the isolated organelles, thus impacting functional analysis.

Microfluidic technologies offer enhanced precision in organelle isolation from sparingly available starting samples and can overcome limitations of isolated-organelle yield and sample-prep throughput. These tools include techniques utilizing magnetic nanoparticles [13], immuno-affinity [14, 15], flow-based or channel structures [16-19], digital microfluidics [20, 21], magnetophoretic-based microfluidics [22], and devices structured to capture DNA [23, 24]. While precise, multi-step process flows (e.g., on-chip extraction, isolation, and off-chip recovery) can be a source of organelle damage and yield loss.

Even with the advent of precision tools for organelle isolation, the post-isolation pooling of isolated organelles remains ubiquitous, making even contemporary microfluidic techniques incompatible with follow-on single-cell or single-organelle analyses that require indexing of organelle back to the originating cell. Indexing an isolated organelle to the originating cell forms a basis for understanding organelle-derived heterogeneity that exists between cell types and among individual cells, even of the same type [25]. In a related aspect of performance: the preservation of spatial information is increasingly sought, such as mapping an isolated organelle(s) back to the originating tissue context. Logically, mapping back to the single originating cell is also sought because functional links between biological processes can (and do) occur at the level of single cells.

An active area of organelle- and cellular-level biology is the study of the nucleus as a coordinating—and typically the largest—cellular organelle. Microfluidic tools make single-nucleus measurements possible. To analyze chromosomal DNA from single nuclei, Benítez et al. introduced a micropillar array and hydrodynamic flows to extract and stretch chromosomal DNA from ~100 single mammalian cells per chip [26]. Following these precise, in-situ assays of chromosomal-DNA stretching from a single cell, DNA was recovered and quantified off chip. Similarly, Wang et al. utilized microchannel geometries to isolate and stretch chromosomal DNA from 10-20 nuclei for subsequent DNA fluorescence in-situ hybridization (FISH), with each signal traced back to its originating nucleus [27]. While limited to assessing DNA damage, conventional agarose-slab embedded and microwell-based comet assays do allow researchers to assess DNA damage and map back to originating cell [28]. These existing techniques point to the promise for assessing other nuclear components—including proteins such as transcription factors—and mapping said measurements back to the originating cell and/or tissue context.

With a focus on introducing tools for single-cell resolution protein measurement, a suite of single-cell immunoblotting modalities designed using microwell-isolated single cells, including single-cell western blots [29-31] is introduced. With an eye towards organelle-biology and subcellular omics, tools for single-nucleus isolation using microwell-isolated mammalian cells are introduced subjected to differential detergent fractionation (DDF), a technique that employs a sequence of detergents with varying solubilization strengths to selectively extract and separate cellular components based on membrane properties [2, 32]. In one example of a single-nucleus resolution analysis made possible by combining microfluidic precision with DDF, a single-cell Western blot was performed of each cytoplasmic compartment and a distinct electrophoresis of each nuclear compartment for an array of cells. In a second example that extends on the assay just described, the Western blot analysis of each single nucleus was swapped out with a PCR assay, allowing both cytoplasmic protein targets and nuclear DNA and RNA targets to be detected in the same originating cell [33-35]. While both single-nucleus precision assays are suitable for sparingly available starting specimens (<10 starting cells, e.g., isolated circulating tumor cells, individual blastomeres comprising two-and four-cell preimplantation murine embryos), sample and analysis throughput must be increased for applicability to larger-cell-number specimens.

Here, a single-cell resolution organelle isolation method is introduced incorporating a single-cell isolation via a polyacrylamide microwell array that is optimized for nuclear isolation after DDF. Microfluidic automation was utilized to enhance throughput while offering the capacity to isolate and then index individual nuclei back to each originating cell. In a multi-layered, planar microfluidic device, individual cells are isolated by settling into an array of polyacrylamide microwells, one cell per microwell. Perturbation, proteomics, or imaging analysis can be performed on intact cells in these top-layer polyacrylamide gel microwells. To isolate and extract single nuclei for further analysis, each cell's cytoplasmic membrane is lysed using DDF, and one intact nucleus remains in each microwell. To concurrently transfer each nucleus to an aligned PDMS microwell situated below the PAG microwell, an interleaving layer of through holes filled with a dissolvable gel is actuated. These dissolvable ‘trapdoor’ in the floor of each PAG microwell opens when the dissolvable gel is exposed to reducing agents (i.e., dithiothreitol (DTT)) and suction is applied using an attached microfluidic vacuum manifold. Once the trapdoors are open, 100's of nuclei are simultaneously transferred from the PAG microwell array to the PDMS microwell array, at one nucleus per microwell occupancy. Here, the multi-layer fluidic design, chemical and hydrodynamic control optimization, and resultant organelle isolation and extraction performance of this single-nucleus isolation and extraction technique are detailed in the following examples.

### Example 1—Fabrication of VacTrap

First, the BAC-gel is polymerized atop a through-hole glass slide, creating a stable, covalently bonded trapdoor. After the BAC-gel polymerizes, the Bis-gel is then polymerized directly on top of the BAC-gel, forming the microwells with the BAC-gel as a base of the microwell. This layered assembly allows for efficient isolation of intact mammalian cells for downstream applications, such as perturbation, imaging, or proteomics analysis. The through-hole glass slide acts as a gate for isolating the nucleus from each cell, providing structural support throughout the single-cell handling steps and ensuring stability during the alignment to the PDMS nucleus-receiving microwells. Without the support of the glass, the thin composite of BAC and Bis-gels (~100 μm thick) would collapse during the dissolution process. Additionally, the through-hole glass slide forms a well-defined path for nuclei to travel from the Bis-gel microwells, through the trapdoor, and into the PDMS microwells. This setup physically transfers each nucleus into a PDMS compartment compatible of with standard biochemical processes, such as PCR. Glass is an ideal material for this design due to its strong bonding properties with both polyacrylamide and PDMS, which is commonly used in single-cell and molecule analyses. Its ability to form stable bonds with both polyacrylamide and PDMS makes it optimal for this system.

The trapdoor at the base of each top-layer Bis-gel microwell is designed to be initially closed, to open with chemical and mechanical triggers, and then remain open (as seen in the schematics of FIGS. 1B-1C). To achieve these functions, the trapdoor is composed of a layer of N,N′-bis(acryloyl)cystamine (BAC), a reversible crosslinker, polymerized with acrylamide monomer to form a dissolvable polyacrylamide gel layer (BAC-gel), cast on a 400-μm thick glass slide with laser-etched 100-μm diameter through holes (FIGS. 2A and C) [36]. Application of DTT results in the degradation of the disulfide-cross-linked BAC-gel due to the thiol-disulfide exchange reaction (seen in the schematic of FIG. 2B) [37-39]. Application of a suction force to the bottom of the PDMS microwell receiving layer transfers force up to the sandwiched trapdoors and initiates nuclei transfer from the Bis-gel microwells into said PDMS receiving microwells. To be effective at transmitting the suction force from the bottom of the PDMS microwells to the trapdoors, the receiving PDMS microwells are designed with ultra-thin (~40 μm) bases (floors). With the vacuum manifold mated to the bottom of the multi-layer assembly, the PDMS microwell floor flexes outward upon application of suction and material is pulled—via the trapdoor—from the top Bis-gel microwell into the receiving PDMS microwell (seen in the schematic of FIG. 1C).

100-μm diameter, 400-μm thick through-hole glass slides (28 mm by 40 mm) were generously provided by Arralyze (LPKF Laser & Electronics AG, Germany). BOROFLOAT® 33 (Schott AG), a borosilicate glass that is widely used for life science applications due to low autofluorescence and high optical transparency in the visible region, was used as a substrate. Details on the Laser Induced Deep Etching protocol can be found in previous studies [36]. The slides were silanized to enhance the gel bonding on the glass slide by adding a methacrylate group as previously described [30].

BAC-gel fabrication was performed in a glove bag (Thermo Scientific, 093737.LK) with continuous nitrogen flow to prevent oxygen inhibition of polymerization. 50-μm Kapton tape rails (315-CQT-0.250-ND) were taped onto a large glass slide (Ted Pella, 260234-25) with 20 mm spacing to align the through-hole area. The glass slide was washed with IPA and dried with nitrogen. Gel slickR (Lonza) (600 μL) was spread between the Kapton tape rails and dried at room temperature. The glass slide was then washed with water and dried with a Kimwipe using a buffing motion to remove excess gel slick.

A 20 mm×18 mm PDMS membrane was cut and applied to one side of the through-hole glass slide to limit gel precursor diffusion during BAC-gel fabrication. The through-hole glass slide was taped atop the Kapton tape rails on the large glass slide with the PDMS membrane facing up. At least ~5 mm from each long side of the through-hole glass slide should sit on top of the Kapton tape rails, resulting in the gel-free edges after fabrication. The assembly was degassed for 10 min before being moved to a glove bag until the gel precursor was ready.

10% APS (w/v) and 10% TEMED (v/v) were prepared with molecular biology grade water and moved to the glove bag. BAC solution was made by dissolving ~22 mg of BAC in 100% methanol, followed by vortexing. BAC-gel precursor was prepared with 6% (w/v) acrylamide, 1×Tris-Glycine (pH 8.3), molecular grade water, and various concentrations of BAC (0.150%, 0.200%, 0.250%, 0.350%, and 0.400%). For some experiments, 100 mM fluorescein o-acrylate in DMSO was added to the gel precursor for a final concentration of 0.2 mM. The gel precursor was degassed and sonicated for 15 min before adding 10% APS and 10% TEMED at a final concentration of 0.1% under the glove bag. 1 mL of gel precursor was quickly wicked through the through-hole glass slide and polymerized under nitrogen for 20 min. After polymerization, the through-hole glass slides and the PDMS membrane were removed, and the gel was incubated with DI water for 5 min before removal from the rails. The gels were kept in water for at least 2 hours before use.

A customize 8×8 photomask (Artnet Pro) with 40-μm-diameter dark circular features and transparent fields was affixed to heat-resistant borosilicate glass (8″×6″, ⅛″ thickness, McMaster Carr 8476K72). Two pieces of 60-μm-thick Kapton tape (3M 5419) were applied to form two rails for Bis-gel fabrication, set to cover the through-holes and BAC-gel area.

Gel slick (400 μL) was applied between the rails and dried at room temperature for 3 min. Excess gel slick on the mask was cleaned with a Kimwipe using a circular buffing motion. On the other side of the glass plate, a long pass filter sheet (8″×6″) was cut and fixed with Kapton tape.

The BAC-gel was dried with nitrogen before attaching a 20 mm×18 mm PDMS membrane to cover the through-hole. Two pieces of 50-μm Kapton tape were applied to the gel-free edges (~5 mm wide) of the through-hole glass slide and cut to shape. These rails compensate for BAC-gel height expansion when exposed to the Bis-gel precursor. The entire assembly was moved to a vacuum chamber and kept closed without vacuum until the gel precursor was ready.

VA-086 photoinitiator was dissolved in water to a final concentration of 2% (w/v). Bis-gel precursor was prepared with molecular grade water, 7% Acrylamide/Bis-acrylamide (29:1), 3 mM BPMAC in DMSO, 1×Tris-glycine (pH 8.3), and 1% VA-086, adjusted with molecular biology grade water. To stain the gel, 100 mM Rhodamine B methacrylate was added to the precursor for a final concentration of 0.2 mM. The gel precursor was degassed for 10 min before wicking through the BAC-gel through-hole glass slide and vacuuming until all bubbles were removed.

The glass plate was then placed under an OAI UV exposure system (Optical Associates, Incorporated) with UV power of ~20 mW/cm² (OAI UV Probe 365 nm, measured without the long-pass filter) for doses of 1400, 1600, 1700, 1800, and 2000 mJ/cm². The standard dose for most experiments was 1700 mJ/cm² (~85 s exposure with ~20 mW/cm² UV energy). After photopolymerization, gels were incubated with water for 5 min before detachment. The composite gels were kept in molecular biology grade water until use.

Alignment strategy for fabrication of the multi-layered, interconnected VacTrap. For nucleus transfer to be successful, 40-μm diameter top-layer Bis-gel microwell must be polymerized and aligned atop of each 100-μm diameter glass through hole coated with 50 μm-thick layer of the BAC-gel that will function as a trapdoor conduit to the receiving 250 by 350-μm PDMS microwell (FIG. 2C). The BAC-gel here will act as a temporary base of the Bis-gel microwell. Alignment must be achieved to sufficient precision across the 15 by 15 mm, 256 Bis-gel microwell array. One time-sensitive constraint arises: How to delay the polymerization and formation process of the Bis-gel microwell until their location is defined to align with each through-hole of the glass slide?

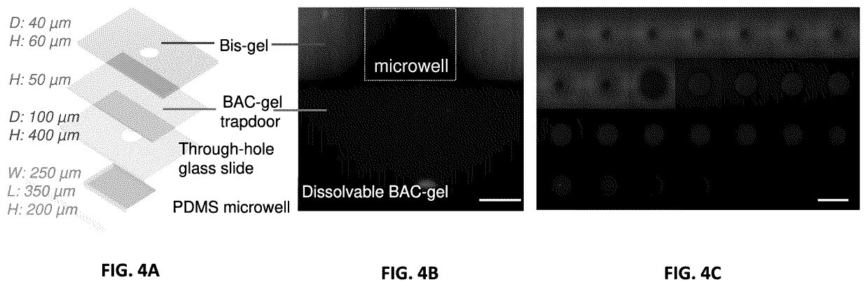

To achieve this balance of timing, we implemented two distinct polymerization methods for BAC-gel and Bis-gel, each having a different time constant for polymerization, seen in FIGS. 4A-4C. Since there is no restriction to the location or polymerization time of the BAC-gel, we employed the chemical polymerization for the BAC-gel layer. The BAC-gel was polymerized on top of the through-hole glass slide using acrylamide monomers and BAC as a cross-linker through free radical polymerization with tetramethylethylenediamine (TEMED) and Ammonium Persulfate (APS). To precisely position the Bis-gel microwells directly over the BAC-gel-coated through holes—ensuring the effective transfer of nuclei across multiple layers of the VacTrap system—the Bis-gel was photopolymerized using 2,2-Azobis[2-methyl-N-(2-hydroxyethyl)pro-pionamide] (VA-086, 1%) as a photoinitiator [40] A photomask was employed to define the microwells diameter and location. Under UV exposure, the Bis-gel precursor in the transparent regions of the mask was exposed and polymerized, while the opaque regions (containing 256 circular features, each 40 μm in diameter) blocked UV light, preventing polymerization and forming the microwells (FIG. 2C). This photopolymerization approach allowed sufficient time to align the mask with the through holes, ensuring the Bis-gel microwells were accurately positioned over the BAC-gel-coated through holes, forming a composite gel. To initiate the transfer, the composite gel was then aligned to the PDMS microwell and the vacuum manifold using brightfield microscopy. Our vacuum manifold utilizes trapezoid pillars (surrounding each PDMS microwell) to prevent the PDMS microwell from collapsing when a vacuum force is applied. This configuration ensures continued contact between the through-hole glass slide and the PDMS microwell.

### Example 2: Design and Fabrication of the Trapdoor Features

The diameter of the top-layer whole-cell receiving Bis-gel microwells is designed to closely match the diameter of individual mammalian cells (~30-40 μm). To achieve an aspect ratio (1.3) designed to reduce the likelihood of capturing multiple mammalian cells in each microwell, 60-um tall Bis-gel microwells were fabricated [30].

To enhance the cell-settling efficiency, the Bis-gel whole-cell receiving layer is dehydrated prior to introducing a cell suspension. Drying polyacrylamide microwell results in the microwell diameter expanding upon dehydration by ~1.5× for gels chemically polymerized (e.g., TEMED, APS). Deviations from an aspect ratio of ~1.3 lead to >1 cell per microwell occupancy, which is not desired in single-cell resolution assays or sample preparation. Consequently, for a photopolymerization (versus chemical polymerization) process, we sought to understand the effect of UV dose (energy×exposure duration) on photopolymerization of the Bis-gel atop the dissolvable BAC-gel layer.

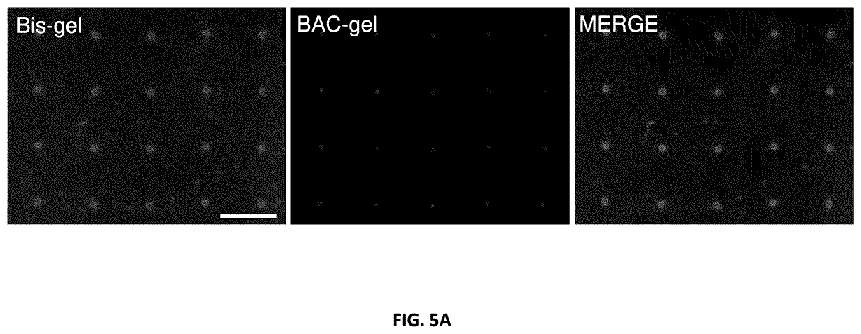

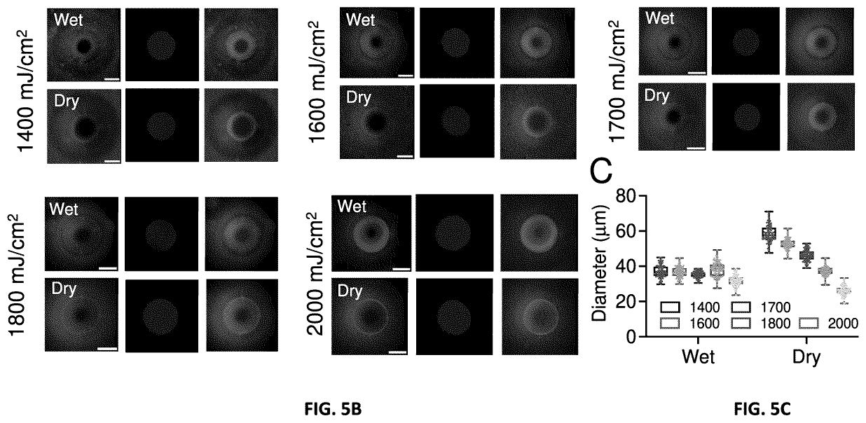

It was set out to determine what range of UV doses minimize Bis-gel expansion after dehydration, while preserving a target hydrated Bis-gel microwell diameter of 40 μm. All the while, the process maintains the Bis-gel layer as co-planar on top of the polymerized dissolvable BAC-gel in such a way that (1) the BAC-gel fully covers the top of the glass through holes and (2) the Bis-gel microwells are each aligned with the through holes in the glass slide (FIG. 5A). Across a wide UV-dose range (1400-2000 mJ/cm²), a ~1.5× expansion in diameter for the Bis-gel microwells after dehydration was measured when polymerizing using the lowest UV doses (1400 mJ/cm² and 1600 mJ/cm²) (FIG. 5C). At 1400 mJ/cm², we observed darkening beneath the microwells by brightfield microscopy, particularly when approaching the through-hole glass slide during a z-axis sweep. We attributed the observation to potential under-polymerization of the Bis-gel, as indicated by a 57% increase in diameter after dehydration (Øhydrated=37.2±3.5 μm, Ødehydrated=58.6±5.0 μm, N=100) (FIG. 5C). In contrast, a higher UV dose of 2000 mJ/cm² resulted in a 25% shrinkage of the microwell diameter, with both hydrated and dehydrated microwells being too small for single-cell encapsulation (Øhydrated=31.7±3.1 μm, Ødehydrated=26.1±2.8 μm, N=100). Optimal results were achieved at a UV dose of 1700 mJ/cm², where the diameter of 100 microwells was measured at Øhydrated=35.3±1.8 μm and Ødehydrated=46.1±3.3 μm, thus maintaining the target microwell diameter of ~32 and 40 μm before and after drying, respectively, as is suitable for single mammalian cell encapsulation.

Based on these observations, it is hypothesized that increasing the UV dose increases the Bis-gel stiffness and, thus, reduces the susceptibility of Bis-gel microwells to expansion upon dehydration. Previous research by Sheth et al [41] determined that the Young's modulus—a measure of the stiffness of a hydrogel—is directly proportional to UV dose. The study considered photopolymerization of polyacrylamide hydrogels with the photoinitator Irgacure 2959 across a UV-dose range of 1500-2600 mJ/cm². The proposed underlying mechanism implicates higher UV dose to enhanced crosslinking reactions, resulting in formation of more functional crosslinks in the resultant gel versus those observed in a lower UV dose process. The crosslinks increase gel stiffness and make the hydrogel less likely to shrink upon dehydration.

The PDMS microwell array consists of 16 rows by 16 columns of rectangular microwells, each measuring 350 μm by 250 μm, with a 1 mm spacing center-to-center. The microwell SU-8 mastermold was fabricated using SU-8 2100 (Kayaku Advanced Materials) to achieve a height of 200 μm, following the manufacturer's instructions. Then the PDMS microwells were produced by spinning approximately 5 g of a 10:1 PDMS mixture on the SU8 mastermold in two steps: the first step for 5 seconds at 100 rpm with an acceleration time of 5 seconds, and the second step for 30 seconds at 400 rpm with an acceleration of 100 rpm, followed by 3 hours curing at 80° C. Before any experiment, the PDMS microwell was deposited in the air plasma cleaner (PDC-32G, Harris plasma) with a vacuum setup of 0.470 torr using an ICME vacuum pump. The radiofrequency power was set to High for 3 minutes.

A vacuum manifold was prepared by casting approximately 27 g of a 10:1 PDMS mixture onto a 100 μm height SU-8 mold using SU-8 3050 (Kayaku Advanced Materials), also according to the manufacturer's instructions. The vacuum manifold featured trapezoid structures with bases of 250 μm and 443 μm, and legs of 147 μm. Prior to PDMS casting, the PDMS mixture was degassed for 1 hour before being applied to the wafers. All PDMS was cured at 80° C. for 3 hours and allowed to cool to room temperature before use. The vacuum manifold had four outlets, which were created using a 2.5 mm biopsy punch (Integra) and connected to soft PVC Plastic Tubing for Air and Water, 1/32″ ID, 3/32″ OD (McMaster-Carr) for vacuum application.

Before aligning the device, the composite gel was gently dried using nitrogen. The back of the glass slide was then cleaned with Scotch tape to ensure a seamless contact between the PDMS microwell and the composite gel. The composite gel was initially aligned with the PDMS microwell, then inverted, and the vacuum manifold was carefully applied to the back of the PDMS microwell.

### Example 3—Chemico-Mechanical Actuation of Trapdoors to Open Fluidic Connection Between Stacked Microwell Layers

It was next sought to identify chemical and mechanical conditions well suited to actuating physical transfer of a nucleus through each trapdoor feature. Previous research has reported 10-20 mM DTT dissolving 0.392-0.500% BAC-gels in 1-5 min [39, 42]. However, these previous studies have considered dissolution of BAC-gel in a bulk form or as BAC-gel droplets immersed in a DTT solution, with thermoshaking. [39, 42]. The layered microfluidic device presents a materially different dissolution environment for DTT-actuated BAC-gel dissolution. In our layered system, DTT must diffuse from point of application, through a 60-μm deep Bis-gel microwell, and then dissolve the 50-μm thick BAC-gel layer from the top.

In tandem with considerations of the BAC-gel composition, compatible approaches were considered to apply force to the dissolving BAC-gel and expedite formation of a fluidic interconnection between the two layers. Primary among the considerations was a vacuum-driven force wherein a microfluidic vacuum manifold was attached underneath the nucleus-receiving PDMS microwell layer. PDMS casting fabricated a thin PDMS floor in each nuclei-receiving PDMS microwell (thickness ~40 μm). The thin PDMS floor is important to provide physical compliance sufficient to effectively transfer vacuum-generated suction force from the vacuum manifold layer to the contents of the PDMS microwell, the BAC-gel in the glass through holes, and finally into the upper Bis-gel microwell compartment. The vacuum manifold generates continuous negative pressure across the gas-permeable PDMS thin floor, allowing air to diffuse from the Bis-gel microwell through the BAC-gel and glass through-holes, which in turn drives DTT flow through the Bis-gel microwell, efficiently dissolving the BAC-gel.

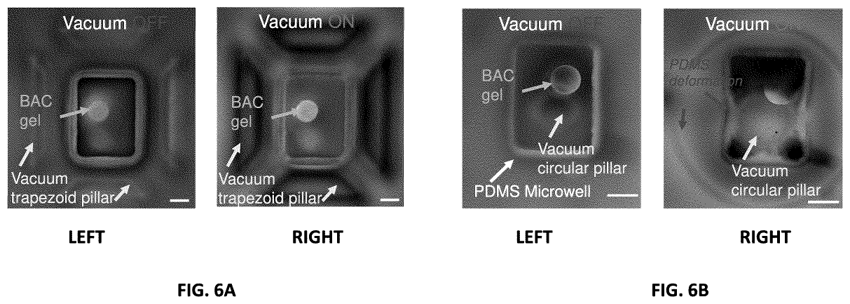

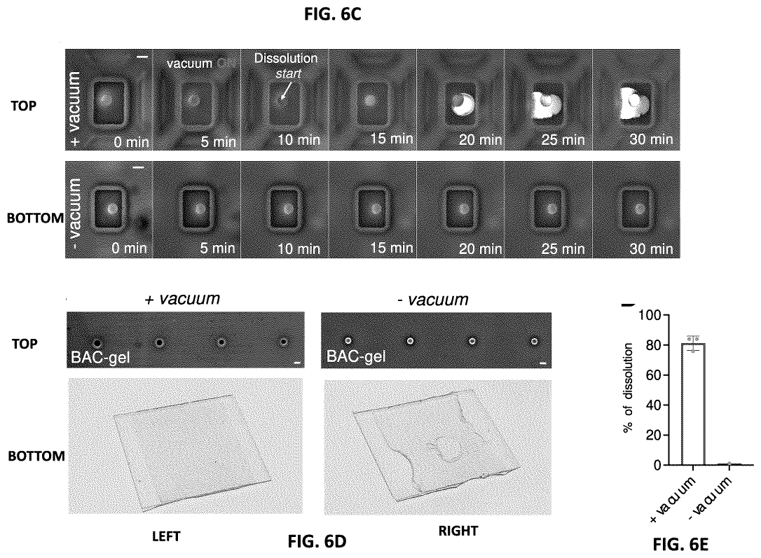

To prevent collapse of the PDMS microwell and floor when vacuum force is applied, an array of structural-support pillars surrounding each PDMS microwell was employed, thereby ensuring supportive structural contact between the through-hole glass slide and the PDMS microwell layer (FIG. 6A). First, circular cross-section pillars were employed and physical behavior and features were observed when the BAC-gel layer incorporated a fluorescently labeled acrylamide monomer. However, it was found that the circular cross-section pillars did not prevent PDMS microwell deformation under application of the vacuum force (FIG. 6B). Circular cross-section pillars resulted in detachment between the PDMS microwell and the vacuum manifold. In contrast, when the larger-surface area trapezoidal cross-section pillars were employed, the PDMS microwell was not observed to either deform or detach, and fluidic connectivity was observed between the stacked Bis-gel and PDMS microwells. Consequently, trapezoidal cross-section support pillars were utilized around the PDMS microwells. To understand the importance of not just dissolving the BAC-gel comprising the trapdoor, but also applying a gentle suction force on that depolymerized BAG-gel bolus, the dissolution process was conserved with and without applied vacuum from the vacuum manifold (FIG. 4B) for a BAC-gel trapdoor fabricated with 0.2% BAC crosslinker, using 40 mM DTT. With the vacuum applied, dissolution through a 50-μm-thick BAC-gel layer occurred within 10 min (FIG. 6C, top). Upon activation of the vacuum, the DTT rapidly reached the microwell, indicated by the reduction in fluorescence signal from the BAC-gel on top of the through-hole glass. Within the subsequent 5 min, dissolution commenced. Dissolution was considered complete when the fluorescence signal from the BAC-gel reached its maximum before a reduction in fluorescence. The signal trend indicates that the dissolved fluorescent BAC-gel initially accumulates at the glass through holes, resulting in a peak fluorescence signal. As the gel continues to dissolve, the liquified gel passes through the through-hole and into the underlying PDMS microwell, causing a decrease in fluorescence as the material moves out of the focal plane. With the vacuum support, the BAC-gel around the through holes is fully dissolved, indicated by the loss of fluorescence in the through-hole area (FIG. 6C, bottom). In contrast, when a trapdoor feature composed of 0.2% BAC was exposed to 40 mM DTT without application of an external mechanical force (-vacuum), the BAC-gel layer did not dissolve and transfer into the PDMS microwell, and no fluidic nor materials connection was observed between the stacked Bis-gel and PDMS microwells after 30 min (FIG. 6C), as evidenced by the fluorescence signal of BAC-gel remaining around the through-hole (FIG. 6C). The vacuum facilitated dissolution in an average of 80% of microwells (~179 microwells). Without applied vacuum, dissolution of the BAC-gel was observed in less than 1% of microwells (FIG. 6D). Dissolution efficiency depends on the precise alignment of the Bis-gel microwell and trapdoor feature with the lower-layer PDMS microwell to ensure the suction force is transmitted effectively through the microwell stack. Ensuring timely dissolution of the BAC-gel is crucial to maintaining the integrity of the Bis-gel microwell. Without specific dissolution within the through-hole area only, the Bis-gel can detach due to a loss of structural support from the BAC-gel and the through-hole glass slide (FIG. 6D, left and right, FIG. 6E). These observations suggest the significance of applying suction to facilitate fluidic interconnection between the stacked microwell layers.

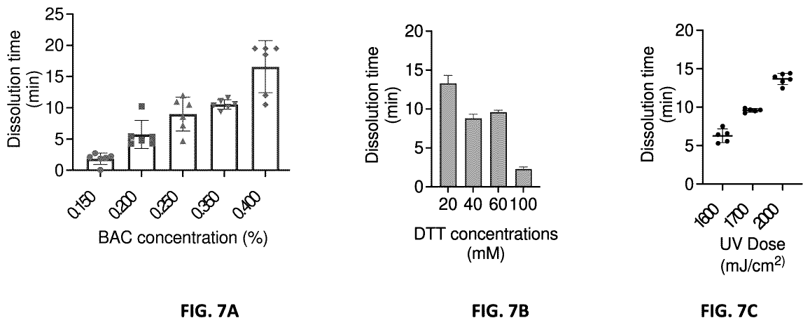

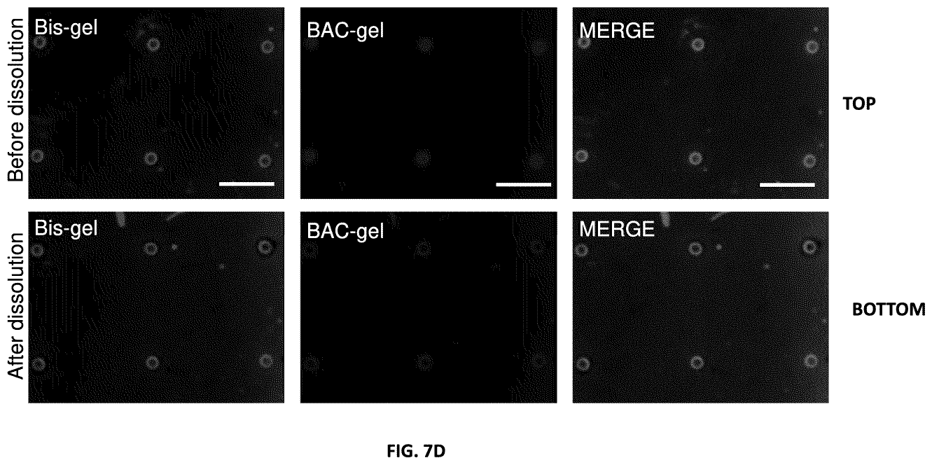

To understand the practical implications of dissolving a BAC-gel in a layered configuration, parameters were studied that influence the dissolution rate, including BAC concentration, DTT concentration, and UV dose used in Bis-gel photopolymerization (FIG. 7A). First a range of BAC crosslinker concentrations from 0.150% to 0.400% were tested. Higher BAC concentrations resulted in a stiffer gel exhibiting a longer time to dissolve. Therefore, a low BAC concentration to facilitate rapid dissolution of the BAC-gel layer was pursued while still maintaining the integrity of the Bis-gel microwell and dissolvable trapdoor feature. A 50-μm thick BAC-gel with 0.150% BAC can be dissolved by 100 mM DTT in 3 min was observed (FIG. 7A), which was in the target dissolution-performance range. However, this lower BAC concentration made the gel more susceptible to tearing during the fabrication process. With that in mind, a 0.200% BAC was observed to dissolve within 3-5 min using 100 mM DTT (FIG. 7A), still within the desired timeframe but with enhanced mechanical robustness which is helpful for reliable fabrication. Taken together, a 0.2% BAC-gel was selected for further analysis.

The four outlets of the vacuum manifold were connected to tubing and a house vacuum system. Subsequently, 300 μL of DTT was delicately applied to the surface of the gel, followed by a 2-minute incubation period before activating the vacuum.

To assess nuclei transfer between the composite gel and PDMS microwell arrays, the nuclei were allowed to gently settle onto the gel for 10-15 min. Afterward, the gel was washed with PBS to remove excess nuclei, followed by the application of DTT and the activation of the vacuum.

In tandem, we considered a range of DTT concentrations from 20 mM to 100 mM for dissolution of trapdoor features fabricated with 0.2% BAC-gel, with complete dissolution achieved in 3 min with 100 mM DTT. Application of 20 mM of DTT required nearly 15 min for dissolution (FIG. 7B). However, DTT is a common redox reagent used to break down protein disulfide bonds, including antibodies [43]. Therefore, for potential proteomics applications in the PAG microwell layer, we sought to reduce DTT concentrations to 40 mM, followed by several washes with high pH buffer (>8) at high temperature to deactivate DTT before immunoprobing. DTT does not interfere with PCR or reverse transcription, making this dissolvable gel suitable for common genomic and nucleic acids applications such as DNA or RNA-seq [39].

Surprisingly, a UV dose used for Bis-gel photopolymerization was found that did affect the dissolution of the underlying BAC-gel trapdoor, with increasing UV dose increasing the required dissolution time (FIG. 7C). However, choosing a low dose of UV for Bis-gel photopolymerization could lead to underexposure causing microwell expansion and incomplete polymerization beneath the Bis-gel microwell. It was hypothesized that UV-based activation homolytically cleaves disulfide bonds to yield two separated thiol radicals [44, 45]. While disulfide bonds could reform if the radical species generated remain in proximity after cleavage, the radicals may recombine with different thiol radicals within the gel matrix, not necessarily from the same original disulfide bridge. Such recombination would cause an observed temporal delay in dissolution. Moreover, excess photoinitiator (VA-86) trapped in the Bis-gel may lead to further polymerization of the BAC-gel around the microwell area, causing further delay in dissolution. With 100 mM of DTT, a 0.2% BAC concentration, and a 1700 mJ/cm² UV exposure for the Bis-gel, dissolution was completed in <5 min without any detectable damage to the Bis-gel microwell after dissolution (FIG. 5D).

### Example 4: Actuation of Trapdoor Features Allows Concurrent Physical Transfer of Isolated Nuclei

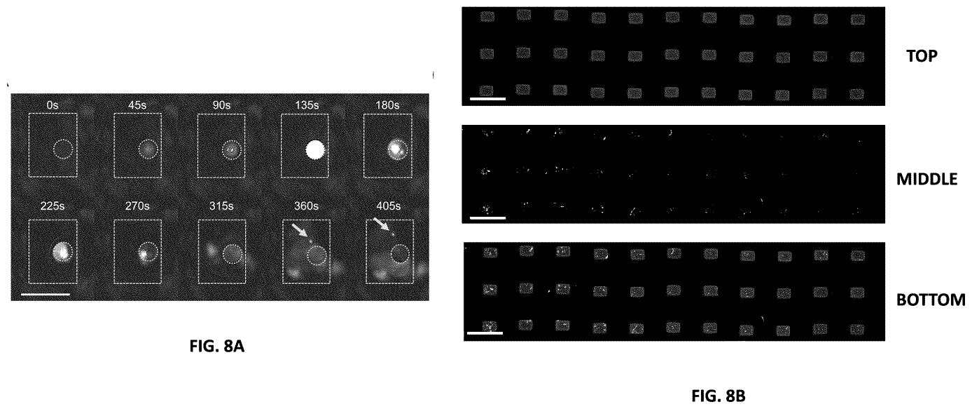

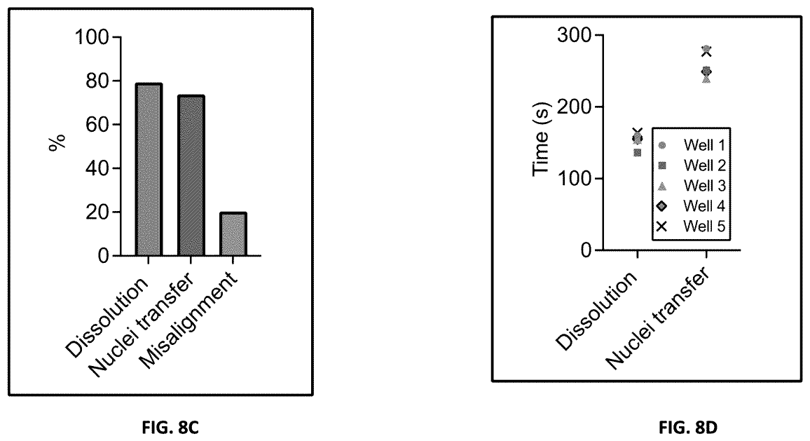

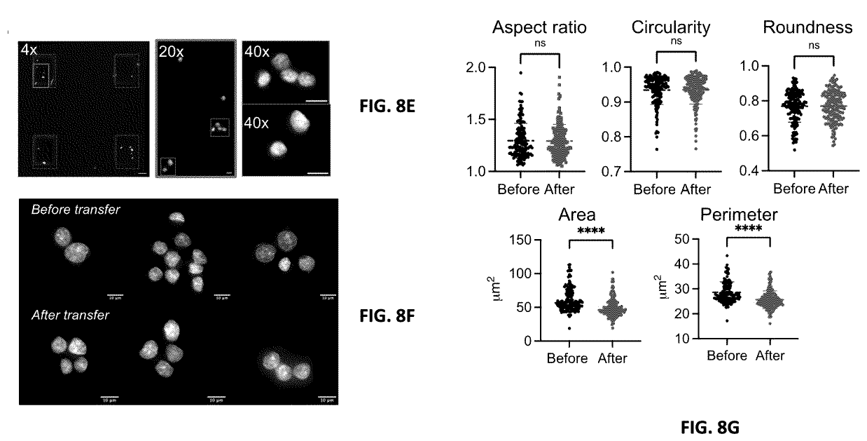

It was next sought to understand the capability of VacTrap to transfer isolated nuclei through the dissolved BAC-gel while maintaining the physical integrity of the nucleus after an applied (suction) mechanical force. To assess simple physical integrity of isolated nuclei, fluorescence microscopy was employed to inspect whether transferred nuclei were physically intact or physically compromised after transfer through a 0.2% BAC-gel trapdoor dissolved by applying 100 mM DTT and suction. FIG. 8A illustrates nucleus transfer through the trapdoor of the BAC-gel into the nuclei-receiving PDMS microwell. Nuclei were observed transferring into the nucleus-receiving PDMS microwells at ~360 s after vacuum activation while the dissolution began first at ~135 s. By fluorescently labeling both the BAC-gel in the trapdoor feature and the isolated nuclei with HOECHST 33342 nuclei transferred in nearly 80% of PDMS microwells inspected was observed (FIGS. 8A and 8B).

To understand the degree of synchronization in the dissolution times across the trapdoor features in a microwell array (FIG. 8D), dissolution was monitored with fluorescence microscopy and measured trapdoor BAC-gel dissolution times ranging from 136-160 s, with an average of 154+/−10 seconds (N=6) with nucleus transfer occurring at ~105+/−15 seconds post-dissolution. The observed delay between the initial dissolution of the BAC-gel trapdoor and nucleus transfer arises from the requirement for complete dissolution of the BAC-gel, which requires 3-5 min with 100 mM DTT. Ensuring simultaneous dissolution and transfer is essential for maintaining the integrity of the nuclei throughout the entire microwell array.

To assess overall yield of trapdoors with suitable performance, inspection of the microwells by microscopy during BAC-gel dissolution revealed that ~80% of the BAC-gel microwells dissolved, corresponding with the percentage of nuclei transferred within the same microwell array (FIG. 8C). The high but not perfect yield in functional trapdoor features is attributed to misalignment between the stacked pair of Bis-gel and PDMS microwell arrays. Additionally, PDMS is known to shrink when cured at high temperatures such as those used in this study, so curing PDMS microwells at room temperature should reduce shrinkage and enhance alignment accuracy.

### Example 5: Transfer of Isolated Nuclei From Single Mammalian Cells

Finally, to extend understanding beyond the physical integrity of extracted nuclei, we sought to assess nuclear phenotype (e.g., morphology). Here, the transparency of the PDMS microwells was leveraged to assess the morphology of nuclei before and after transfer (FIG. 8E-8G). Common morphological parameters including nuclear aspect ratio, circularity, roughness, area, and perimeter were analyzed (FIG. 8G). Our results indicate no detectable changes in aspect ratio, circularity, or roughness before and after nuclei transfer. However, alterations in area and perimeter were observed. It is hypothesized that the changes in area and perimeter are attributable to the response of nuclei upon exposure to DTT during the transfer process as well as imaging artifacts that arise from imaging through the PDMS microwells. Nevertheless, nuclei remained intact and retained overall shape.

### Example 6: Barcoding Microwells

To enable integration across microfluidic platforms compatible with microwells, a deterministic barcoding strategy was proposed for microwells using magnetic beads. Our method consists of two steps: 1) a microfluidic layer directs the flow of barcoded magnetic beads into microwells, 2) while the beads are kept in place using a magnet positioned underneath the microchannels, the delivery layer is replaced with one containing orthogonal channels. This second layer facilitates the delivery of free oligos creating a unique barcode combination in each microwell (as seen in the schematics of FIGS. 3B-3C).

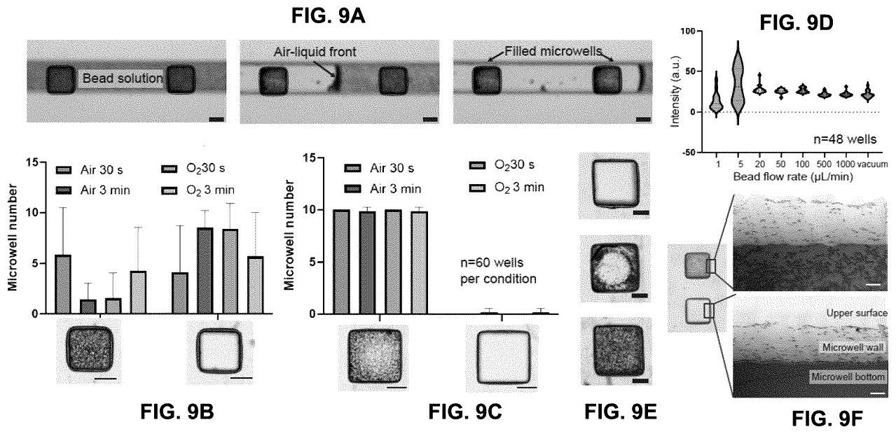

To ensure the efficient and uniform delivery of beads into microwells, the effect of geometries (well size: 200 vs 300 μm) and plasma treatment (O² and air plasma) on microwell filling and bead deposition after evaporation (FIG. 9A-9C, 9E-9F) was evaluated, highlighting the need of larger wells for achieving uniform patterns on the surface. Furthermore, the effect of flow rates (1-1000 μL/min) to the variation in bead deposition was studied. FIG. 9D shows the well-to-well variations of the fluorescent intensity across the tested flow rates, with 100-500 μL/min delivering the lowest variability across 16 microwells.

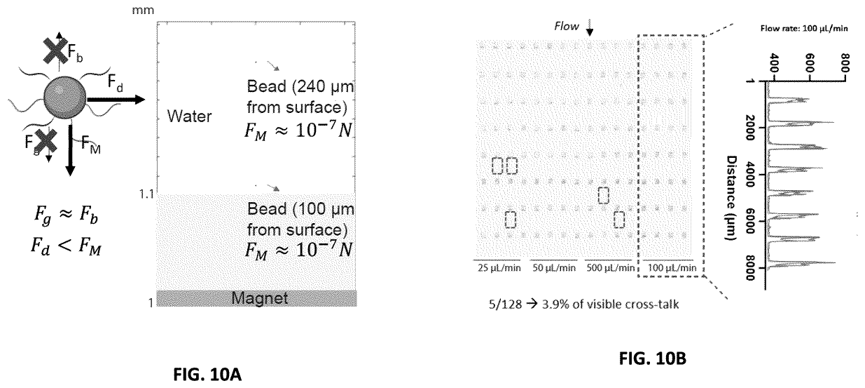

To assess cross-talk after the orthogonal deposition of free oligos, a force balance analysis and a COMSOL simulation was conducted (FIG. 10A). This analysis suggested that the drag force (Fd) experienced by the beads (10-12-10-9N, flow rates 1-500 μL/min) is equal or smaller than the magnetic force (FM) exerted by the permanent magnet (10-9-10-7N). By depositing fluorescently-labeled beads into the microwells at various flow rates, a flow rate of 50-100 μL/min was found that minimizes cross-talk, with 0.7-3.9% of the wells affected (FIG. 10B), comparable to reported 5.5-9.5% [4].

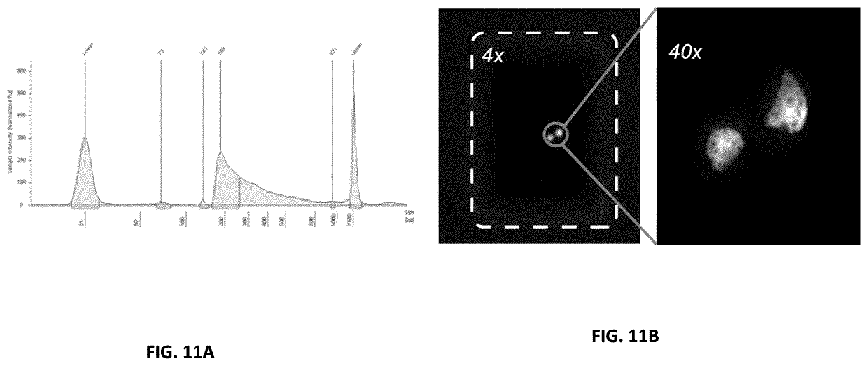

Finally, the effectiveness of the barcoding strategy was demonstrated for scATAC-seq by depositing 16 by 16 unique barcodes to the microwells and conducting the on-chip library preparation. Using 2.5 mg/mL of barcoded beads with 10 μM of free oligos yielded tagmented DNA concentrations of approximately 1.7 ng/μL, meeting most sequencers'library size requirements (FIG. 11A). These microwells are compatible with inverted microscopes for imaging, here shown with stained nuclei as proof of concept (FIG. 11B).

In summary, our proposed barcoding strategy offers a rapid and efficient means to barcode microwells.

Nuclear isolation. Cancer cell line MCF7 Tet-off parental cells were kindly gifted by the Arribas Lab from the Vall' d'Hebron Institute of Oncology. The cell line was authenticated by short tandem repeat profiling by the UC Berkeley Cell Culture facility and tested negative for mycoplasma.For each experiment, cell culture was maintained at 37° C. and 5% CO2 in Dulbecco's Modified Eagle Medium/Nutrient Mixture F-12 (Gibco™ DMEM/F-12, GlutaMAX™ supplement, Thermofisher, 10565018) supplied with 10% fetal bovine serum (Gemini Bio), 0.2 mg/ml Gibco™ Geneticin™ Selective Antibiotic (G418 Sulfate), and 1 μg/ml doxycycline (Sigma) until 80% confluency and detached with 0.05% Trypsin-EDTA (Gibco #25300-054) for 4-5 min.

1 million viable cells were aliquoted into 1.5 ml LoBind Eppendorf tubes. The cells were then centrifuged at 500 g for 5 min at 4° C. After centrifugation, the medium was removed, and the cells were resuspended in 1 ml of cold 1×PBS buffer. The cells were centrifuged again at 500 g for 5 min at 4° C., and the PBS was aspirated. Subsequently, 300 μL of cold lysis buffer was added to the sample, and the cells were mixed 10 times. The sample was incubated on ice for 5 min. After incubation, 1 ml of cold wash buffer was added to each sample, and the tubes were inverted 5 times to mix. The nuclei were pelleted with the hinge facing in at 500 g for 3 min at 4° C., then centrifuged again with the hinge facing out at 500 g for 3 min at 4° C. The supernatant was aspirated in two steps: 1000 μL was removed with a P1000 pipette, and the remaining 50-100 μL was removed with a P200 pipette. The nuclei were gently resuspended in 250 μL of wash buffer using a wide-bore tip (Rainin). The quality and count of the nuclei were assessed using a Countess (10 μL of nuclei with 10 μL of Trypan blue)

To fluorescently label nuclei for imaging, 2 μL of 20 mM Hoechst was added into 1000 μL of PBS to prepare the staining wash buffer. An aliquot of 100,000 nuclei was added to 1000 μL of staining wash buffer and incubated for 20 min on ice. The nuclei were pelleted with the hinge facing in at 500 g for 5 min at 4° C., then centrifuged again with the hinge facing out at 500 g for 5 min at 4° C. The supernatant was aspirated, and the nuclei were resuspended in 1000 μL of PBS to achieve a concentration of approximately 100 nuclei/μL.

### Example 7: Complete scBlot-seq Assay From Mcf7 Single Cells

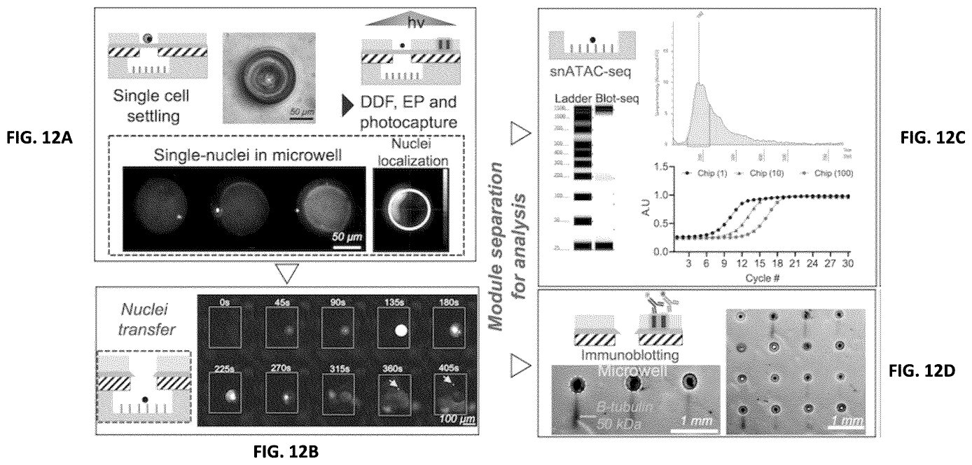

FIGS. 12A-12D depict representative images and graphs demonstrating the complete assay, using MCF7 cells. Single cells were first settled to the composite gel (as seen in FIG. 12A) and undergo scWB, separating cytoplasmic protein but preserving the nuclei by DDF. Then nuclei are transferred by DTT and vacuum-driven force (yellow arrows show nuclei are transferred to microwell) as seen in FIG. 12B. After this step, the modules are separated into two workflows: the bottom layer undergoes snATAC-seq using a vacuum-driven microfluidic system for reagent delivery, showing the nucleosomal pattern through the electropherogram and qPCR (as seen in FIG. 12D), and the top layer was used for detection of the model β-tubulin in polyacrylamide gel (as seen in FIG. 12C).

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

### REFERENCES

1. Liao, P.C., et al., Isolation of mitochondria from cells and tissues. Methods Cell Biol, 2020. 155: p. 3-31.
2. Drissi, R., M.-L. Dubois, and F.-M. Boisvert, Proteomics methods for subcellular proteome analysis. The FEBS Journal, 2013. 280(22): p. 5626-5634.
3. Andersen, J.S., et al., Directed proteomic analysis of the human nucleolus. Curr Biol, 2002. 12(1): p. 1-11.
4. Taylor, S.W., et al., Characterization of the human heart mitochondrial proteome. Nat Biotechnol, 2003. 21(3): p. 281-6.
5. Satori, C.P., V. Kostal, and E.A. Arriaga, Review on recent advances in the analysis of isolated organelles. Anal Chim Acta, 2012. 753: p. 8-18.
6. Parsons, H.T., S.M.G. Fernández-Niño, and J.L. Heazlewood, Separation of the Plant Golgi Apparatus and Endoplasmic Reticulum by Free-Flow Electrophoresis, in Plant Proteomics: Methods and Protocols, J.V. Jorrin-Novo, et al., Editors. 2014, Humana Press: Totowa, NJ. p. 527-539.
7. Wildgruber, R., et al., Free-flow electrophoresis in proteome sample preparation. PROTEOMICS, 2014. 14(4-5): p. 629-636.
8. Li, C.M., et al., Partial Purification of a Megadalton DNA Replication Complex by Free Flow Electrophoresis. PLoS One, 2016. 11(12): p. e0169259.
9. Ramsby, M.L., G.S. Makowski, and E.A. Khairallah, Differential detergent fractionation of isolated hepatocytes: biochemical, immunochemical and two-dimensional gel electrophoresis characterization of cytoskeletal and noncytoskeletal compartments. Electrophoresis, 1994. 15(2): p. 265-77.
10. Sawhney, S., R. Stubbs, and K. Hood, Reproducibility, sensitivity and compatibility of the ProteoExtract subcellular fractionation kit with saturation labeling of laser microdissected tissues. Proteomics, 2009. 9(16): p. 4087-92.
11. Hwang, B., J.H. Lee, and D. Bang, Single-cell RNA sequencing technologies and bioinformatics pipelines. Exp Mol Med, 2018. 50(8): p. 1-14.
12. Butto, T., et al., Nuclei on the Rise: When Nuclei-Based Methods Meet Next-Generation Sequencing. Cells, 2023. 12(7).
13. Sancho-Albero, M., et al., Isolation of exosomes from whole blood by a new microfluidic device: proof of concept application in the diagnosis and monitoring of pancreatic cancer. Journal of Nanobiotechnology, 2020. 18(1): p. 150.
14. Kayo, S., et al., A microfluidic device for immuno-affinity-based separation of mitochondria from cell culture. Lab on a Chip, 2013. 13(22): p. 4467-4475.
15. Takahashi, T., et al., A microfluidic device for isolating intact chromosomes from single mammalian cells and probing their folding stability by controlling solution conditions. Scientific Reports, 2018. 8(1): p. 13684.
16. Lu, H., et al., A Microfabricated Device for Subcellular Organelle Sorting. Analytical Chemistry, 2004. 76(19): p. 5705-5712.
17. Toyama, K., M. Yamada, and M. Seki. Development of microfluidic cell nucleus separator employing rapid chemical treatment. in 2010 International Symposium on Micro-NanoMechatronics and Human Science. 2010.
18. Yamada, M., et al., Millisecond treatment of cells using microfluidic devices via two-step carrier-medium exchange. Lab on a Chip, 2008. 8(5): p. 772-778.
19. Tesauro, C., et al., Isolation of functional mitochondria by inertial microfluidics—a new method to sort intracellular organelles from a small scale biological sample. RSC Advances, 2017. 7(38): p. 23735-23741.
20. Lamanna, J., et al., Digital microfluidic isolation of single cells for-Omics. Nature Communications, 2020. 11(1): p. 5632.
21. Hung, P.-Y., et al., Genomic DNA extraction from whole blood using a digital microfluidic (DMF) platform with magnetic beads. Microsystem Technologies, 2017. 23(2): p. 313-320.
22. Hale, C. and J. Darabi, Magnetophoretic-based microfluidic device for DNA isolation. Biomicrofluidics, 2014. 8(4): p. 044118.
23. Reedy, C.R., et al., Solid phase extraction of DNA from biological samples in a post-based, high surface area poly(methyl methacrylate) (PMMA) microdevice. Lab on a Chip, 2011. 11(9): p. 1603-1611.
24. Lee, C., et al., A Three-Dimensional Printed Inertial Microfluidic Platform for Isolation of Minute Quantities of Vital Mitochondria. Analytical Chemistry, 2022. 94(19): p. 6930-6938.
25. Venkatesan, M., et al., Spatial subcellular organelle networks in single cells. Scientific Reports, 2023. 13(1): p. 5374.
26. Benítez, J.J., et al., Microfluidic extraction, stretching and analysis of human chromosomal DNA from single cells. Lab on a Chip, 2012. 12(22): p. 4848-4854.
27. Wang, X., et al., Microfluidic extraction and stretching of chromosomal DNA from single cell nuclei for DNA fluorescence in situ hybridization. Biomedical Microdevices, 2012. 14(3): p. 443-451.
28. Wood, D.K., et al., Single cell trapping and DNA damage analysis using microwell arrays. Proceedings of the National Academy of Sciences, 2010. 107(22): p. 10008-10013.
29. Hughes, A.J., et al., Single-cell western blotting. Nature Methods, 2014. 11(7): p. 749-755.
30. Kang, C.-C., et al., Single cell-resolution western blotting. Nature Protocols, 2016. 11(8): p. 1508-1530.
31. Yamauchi, K.A. and A.E. Herr, Subcellular western blotting of single cells. Microsystems & Nanoengineering, 2017. 3(1): p. 16079.
32. McCarthy, F.M., et al., Differential Detergent Fractionation for Non-electrophoretic Eukaryote Cell Proteomics. Journal of Proteome Research, 2005. 4(2): p. 316-324.
33. Rosàs-Canyelles, E., et al., Assessing heterogeneity among single embryos and single blastomeres using open microfluidic design. Science Advances. 6(17): p. eaay1751.
34. Rosàs-Canyelles, E., et al., Multimodal detection of protein isoforms and nucleic acids from low starting cell numbers. Lab on a Chip, 2021. 21(12): p. 2427-2436.
35. Rosàs-Canyelles, E., et al., Multimodal detection of protein isoforms and nucleic acids from mouse pre-implantation embryos. Nature Protocols, 2021. 16(2): p. 1062-1088.
36. Sandström, N., et al., Live single cell imaging assays in glass microwells produced by laser-induced deep etching. Lab on a Chip, 2022. 22(11): p. 2107-2121.
37. Bromberg, L., et al., Kinetics of Swelling of Polyether-Modified Poly(acrylic acid) Microgels with Permanent and Degradable Cross-Links. Langmuir, 2005. 21(4): p. 1590-1598.
38. Plunkett, K.N., et al., Swelling Kinetics of Disulfide Cross-Linked Microgels. Macromolecules, 2003. 36(11): p. 3960-3966.
39. Wang, Y., et al., Dissolvable Polyacrylamide Beads for High-Throughput Droplet DNA Barcoding. Advanced Science, 2020. 7(8): p. 1903463.
40. Pan, Q., K.A. Yamauchi, and A.E. Herr, Controlling Dispersion during Single-Cell Polyacrylamide-Gel Electrophoresis in Open Microfluidic Devices. Anal Chem, 2018. 90(22): p. 13419-13426.
41. Sheth, S., et al., UV Dose Governs UV-Polymerized Polyacrylamide Hydrogel Modulus. International Journal of Polymer Science, 2017. 2017: p. 5147482.
42. Takemori, A., et al., BAC-DROP: Rapid Digestion of Proteome Fractionated via Dissolvable Polyacrylamide Gel Electrophoresis and Its Application to Bottom-Up Proteomics Workflow. Journal of Proteome Research, 2021. 20(3): p. 1535-1543.
43. Okuno, T. and N. Kondelis, Evaluation of dithiothreitol (DTT) for inactivation of IgM antibodies. J Clin Pathol, 1978. 31(12): p. 1152-5.
44. Gammelgaard, S.K., et al., Direct Ultraviolet Laser-Induced Reduction of Disulfide Bonds in Insulin and Vasopressin. ACS Omega, 2020. 5(14): p. 7962-7968.
45. Wongkongkathep, P., et al., Enhancing Protein Disulfide Bond Cleavage by UV Excitation and Electron Capture Dissociation for Top-Down Mass Spectrometry. Int J Mass Spectrom, 2015. 390: p. 137-145.
46. Corces, M.R., et al., An improved ATAC-seq protocol reduces background and enables interrogation of frozen tissues. Nat Methods, 2017. 14(10): p. 959-962.

1. A method for isolating cytoplasmic biomolecules and organelles from a single cell in separate chambers, the method comprising:
(a) Isolating one or more single cells in individual first microwells, wherein said first microwells comprise a gel layer and a gel floor layer;
(b) Lysing the one or more single cells to release cytoplasmic biomolecules and organelles;
(c) Applying an electric field to the first microwell in the gel layer, thereby separating cytoplasmic biomolecules within the gel layer;
(d) Collecting organelles in individual second microwells; said collecting comprising (i) dissolving the gel floor layer and (ii) applying a vacuum force;
thereby isolating cytoplasmic biomolecules and organelles from a single cell in separate chambers.

2. The method of claim 1, wherein said isolating of the one or more single cells step in (a) comprises depositing one or more single cells into individual microwells from a microfluidic channel.

3. The method of claim 1, wherein said gel layer comprises one or more of Bis/acrylamide monomer, a photoinitiator, a UV photoimmobilizer, a buffer, and water.

4.-6. (canceled)

7. The method of claim 1, wherein said gel floor layer comprises one or more of a reversible crosslinker, acrylamide, TEMED, and APS.

8.-16. (canceled)

17. The method of claim 1, wherein said electric field is applied under conditions that allow separation of the cytoplasmic biomolecules based on molecular weight (MW) and/or chemical properties.

18.-20. (canceled)

21. The method of claim 1, wherein said dissolving the gel floor comprises contacting the gel floor with a reducing agent.

22.-25. (canceled)

26. The method of claim 1, wherein said second microwell comprises barcoded beads.

27.-29. (canceled)

30. The method of claim 1, wherein said one or more single cells are selected from the group consisting of mammalian cells, human cells, tumor cells, organoids, patient-derived cells or patient-derived organoids, and dissociated human tissues.

31.-32. (canceled)

33. The method of claim 1, wherein said isolating comprises isolating 6, 12, 24, 48, 96, 256, 384 or 1536 single cells in step (a).

34.-35. (canceled)

36. The method of claim 1, wherein the isolating comprises using an apparatus comprising a plurality of first microwells, wherein the first microwells comprise a gel layer and a gel floor layer;
a plurality of second microwells, each first microwell aligned with one of the second microwells,
wherein one or more single cells are to be lysed within the first microwells to release cytoplasmic biomolecules and organelles,
wherein an electric field is to be applied to the first microwell in the gel layer, thereby separating cytoplasmic biomolecules within the gel layer, and
wherein the gel floor layer is to be dissolved to enable organelles to be collected in the individual second microwells.

37. A method for detecting one or more cytoplasmic biomolecules and one or more organelles from a single cell, said method comprising:
(a) isolating cytoplasmic biomolecules and organelles from a single cell according to claim 1;
(b) detecting one or more cytoplasmic biomolecules, said detecting comprising any one or more detection methods comprising electrophoresis, immunoprobing and mass spectrometry;
(c) detecting one or more organelles, said detecting comprising any one or more detection methods comprising sequencing, imaging, morphology identification, in situ hybridization, and rolling circle amplification;
thereby detecting one or more cytoplasmic biomolecules and one or more organelles from a single cell

38.-39. (canceled)

40. The method of claim 37, wherein the organelles are contacted in the second microwell by oligonucleotides corresponding to primer pair sequences under conditions that allow hybridization of the primer pair to nucleic acids of the organelles, thereby forming a complex, and wherein the complex is subjected to amplification and/or sequencing.

41. (canceled)

42. The method of claim 37, wherein the detecting of the one or more cytoplasmic biomolecules is linked to the one or more organelles from the same single cell.

43.-44. (canceled)

45. A method of barcoding microwells suitable for single cell analyses in a microwell system, the method comprising:
(a) labeling magnetic beads with known oligonucleotide barcodes, wherein the oligonucleotide barcodes comprise a bead barcode sequence and a first primer pair sequence;
(b) delivering the labeled magnetic beads into microwells using a microfluidic channel of a first microfluidic device;
(c) removing the first microfluidic device;
(d) applying a magnetic force under conditions that prevent the labeled magnetic beads from escaping the microwells;
(e) delivering free oligonucleotides to the microwells comprising the labeled magnetic beads using a microfluidic channel of a second microfluidic device, wherein the free oligonucleotides comprise a free barcode sequence and a second primer pair sequence.

46.-55. (canceled)

56. A method of highly parallel, dead-end, vacuum-based reagent delivery to microwells suitable for single cell analyses in a microwell system, the method comprising:
(a) bonding a bifurcated delivery membrane to a barcoded PDMS microwell (after organelle transfer to the PDMS microwell);
(b) bonding a vacuum suction layer on top of the delivery membrane; and
(c) activating a vacuum force to generate negative pressure across the delivery membrane, therefore, driving the reagents loading to each microwell simultaneously.

57. An apparatus, comprising:
a plurality of first microwells, wherein the first microwells comprise a gel layer and a gel floor layer;
a plurality of second microwells, each first microwell aligned with one of the second microwells,
wherein one or more single cells are to be lysed within the first microwells to release cytoplasmic biomolecules and organelles,
wherein an electric field is to be applied to the first microwell in the gel layer, thereby separating cytoplasmic biomolecules within the gel layer, and
wherein the gel floor layer is to be dissolved to enable organelles to be collected in the individual second microwells.

58. The apparatus of claim 57, wherein each of the first microwells that is aligned with one of the second microwells form a stacked microwell pair.

59. The apparatus of claim 57, further comprising a plurality of stacked microwell pairs, each microwell pair comprising one of the first microwells and one of the second microwells.

60.-75. (canceled)

76. The apparatus of claim 57, further comprising a glass slide comprising a plurality of through holes, the glass slide positioned between the first microwells and the second microwells, each through hole aligned with one of the first microwells and one of the second microwells.

77. The apparatus of claim 76, further comprising a vacuum manifold layer coupled to the second microwells, the vacuum manifold layer comprising a plurality of pillars surrounding each of the second microwells, wherein a vacuum force is to be applied to the vacuum manifold layer to urge the organelles to move from the first microwells and to be collected in the individual second microwells.

78. The apparatus of claim 77, wherein the second microwells each comprise a second microwell floor layer, wherein an interaction between the pillars and the second microwell floor layer enables substantially continued contact between the glass slide and the second microwells, and prevention of microwell deformation when the vacuum force is applied.

79.-90. (canceled)
